(19) United States
(12) Patent Application Publication
(10) Pub. No.: US 2025/0295619 A1
(43) Pub. Date: Sep. 25, 2025

(54) Use of Salvianolic Acid E in the Preparation of Drugs Targeting Senescent Cells, Inhibiting Tumors or Extending Lifespan

(71) Applicant: Shanghai Institute of Nutrition and Health, Chinese Academy of Sciences, Shanghai (CN)

(72) Inventor: Yu Sun, Shanghai (CN)

(21) Appl. No.: 18/611,114

(22) Filed: Mar. 20, 2024

(51) Int. Cl. A61K 31/195; A61K 31/136; A61K 31/704; A61K 38/14; A61P 35/00; G01N 33/50

(52) U.S. Cl. CPC A61K 31/195; A61K 31/136; A61K 31/704; A61K 38/14; A61P 35/00; G01N 33/5011; G01N 2333/8139

(57) ABSTRACT

The present disclosure provides a use of salvianolic acid E (SAE) in the preparation of drugs targeting senescent cells, inhibiting tumors or prolonging lifespan. The inventors are committed to research in screening drugs targeting tumor microenvironment, enhancing the anti-tumor effect of chemotherapeutic drugs, eliminating senescent cells or restraining cellular senescence. Here, it is revealed that salvianolic acid E exerts its effects by targeting tumor microenvironment and eliminating senescent cells. When combined with chemotherapeutic drugs, it shows extremely significant effects in promoting tumor suppression by removing senescent stromal cells. For a senescence-associated secretory phenotype (SASP), the SAE can also effectively target senescent cells so as to inhibit the SASP. Besides, the SAE can also significantly prolong the life of animals, significantly extend the survival period of the old and improve the life quality of animals.

### TECHNICAL FIELD

The present disclosure belongs to the fields of cell biology and oncology, and more specifically, the present disclosure relates to drugs of inhibiting cell senescence, suppressing tumors or extending lifespan and the use thereof.

### TECHNICAL BACKGROUND

Cell senescence refers to a usually stable and essentially irreversible state of cell cycle arrest in eukaryotic cells, in which proliferating cells become resistant to growth-promoting stimuli, mostly induced by stress signals such as DNA damage. In the 1960s, Leonard Hayflick and Paul Moorhead first described cellular senescence. They observed that human embryonic fibroblasts (WI38) eventually stopped division but remain viable and metabolically active after continuous culture. This phenomenon later became known as replicative senescence, referring to the cessation of continuous proliferation in normal cells after approximately 30-50 divisions (the “Hayflick limit”). Replicative senescence is essentially induced by the progressive shortening of telomeres. During each round of DNA replication, telomeres gradually shorten, eventually reaching a critical length that prevents further replication and thus restrains cell division. Shorter uncapped telomeres elicit a DNA damage response that directly triggers cellular senescence.

Senescent cells are characterized by abnormal morphology, altered metabolic activity, chromatin remodeling, aberrant gene expression, increased lipofuscin, enhanced granularity, severe vacuolization, and a senescence-associated secretory phenotype (SASP) pro-inflammatory phenotype. The concept of SASP was first proposed by Coppe, et. al. in 2008. They found that senescent cells can promote the proliferation of adjacent precancerous cells or increase the malignancy of cancer cells by secreting extracellular matrix proteins, inflammation-related factors and cancer cell growth factors, and collectively referred to as SASP factors. In recent years, numerous studies have indicated that the senescence-associated secretory phenotype (SASP) plays a central pathological role in senescence. Furthermore, the factors secreted by senescent cells can impact surrounding normal cells, while inhibiting SASP has the potential to delay natural senescence. Typical SASP factors include tumor necrosis factor-α (TNF-α), interleukin 6 (IL-6), interleukin 8 (IL-8), interleukin 1a (IL-1a), matrix metalloproteinases (MMP), granulocyte-macrophage colony stimulating factor (GM-CSF), and plasminogen activator inhibitor-1 (PAI1), and so on. These factors promote immune system activation, leading to the clearance of senescent cells and other detrimental factors in the tissue microenvironment, thereby exerting a tumor-suppressive function.

Although various SASP inhibitors known internationally can significantly weaken the SASp, they cannot kill senescent cells in essence. In order to reduce the burden of senescent cells pharmacologically, scientists are developing senescent cell-eliminating drugs, known as “senolytic(s)”. These small molecules, peptides and antibodies can selectively kill senescent cells. Since the discovery of senolytic drugs in 2015, researchers have made considerable progress in identifying other small molecule senolytic drugs and clarifying their competence in senescent cell clearance.

A research has indicated that there is an upregulation of pro-apoptotic pathways in senescent cells and senescent cells rely on senescence-associated anti-apoptotic pathways (SCAPs) to mitigate SASP damage to themselves. This hypothesis has been validated. SCAPs were identified by bioinformatics methods based on expression profiles of radiation-induced senescent human preadipocytes. Some studies have found that senescent cells are dependent on SCAPs through in vitro RNA interference experiments, and identified SCAPs as a vulnerability of senescent cells. This research ultimately led to the discovery of potential senolytic targets within the SCAP network and the discovery of the first generation senolytic drugs, including a combination of dasatinib and quercetin (D+Q). In addition, a study identified an anti-apoptotic protein (BCL-XL) in the BCL-2 family as a SASP component. Following this discovery, a third senolytic drug, navitoclax, was also identified, which is a BCL-2 family inhibitor. Researchers have continued to identify more senolytics, including synthetic small molecules, compounds extracted from natural products, and inhibitors targeting known SCAPs peptides. Furthermore, SCAPs are also being explored as potential senolytic targets.

The SCAPs required for senescent cell survival can vary between cell types. For example, the SCAPs required for survival of senescent human primary adipose progenitor cells differ from those in senescent human embryonic venous endothelial cells (HUVECs). This difference means that drugs targeting a single SCAP may not be able to eliminate multiple senescent cell types. Also, numerous studies have shown that most senolytics are indeed only effective against a limited number of senescent cell types. For example, navitoclax was able to target HUVECs but was ineffective against senescent human adipocytes. Evidence suggests that the efficacy of senolytics may vary even within one specific type of cell. For example, in human lung fibroblasts, navitoclax targets and kills senescent cells in the culture-adapted IMR90 lung fibroblast-like cell line, but is less effective on senescent human primary lung fibroblasts.

Therefore, extensive research is still required to determine the optimal senolytics with broad-spectrum effects. This research will contribute to delaying, preventing or treating various senescence-associated diseases.

### SUMMARY OF THE INVENTION

The aim of the present disclosure is to provide a use of salvianolic acid E (SAE) in the preparation of drugs targeting senescent cells, inhibiting tumors or extending lifespan.

In the first aspect, the present disclosure provides a method for specifically targeted clearance of senescent cells in a tumor microenvironment and tumor inhibition, comprising administering to a subject in need of treatment with a combination of a salvianolic acid E compound or a pharmaceutically acceptable salt, ester, isomer, solvate or prodrug thereof, and a chemotherapeutic drug; wherein the chemotherapeutic drug is capable of inducing a senescence-associated secretory phenotype in the tumor microenvironment after administration.

In other words, the present disclosure also provides a use of a salvianolic acid E compound or a pharmaceutically acceptable salt, ester, isomer, solvate or prodrug thereof, for preparing a composition combined with a chemotherapeutic drug for specifically-targeted clearance of senescent cells in a tumor microenvironment and tumor inhibition; wherein the chemotherapeutic drug is capable of inducing a senescence-associated secretory phenotype in the tumor microenvironment after administration.

In one or more embodiments, the salvianolic acid E is an isolated monomer.

In one or more embodiments, the salvianolic acid E is not present in natural plants, organs, or processed products thereof.

In one or more embodiments, the salvianolic acid E is not present in traditional Chinese medicine.

In one or more embodiments, the tumor is a tumor that exhibits a senescence-associated secretory phenotype in the tumor microenvironment after treatment with genotoxic drugs, and/or is a tumor that develops drug resistance after using genotoxic drugs.

In one or more embodiments, the tumor comprises: prostate cancer, breast cancer, lung cancer, colorectal cancer, gastric cancer, liver cancer, pancreatic cancer, bladder cancer, skin cancer, kidney cancer, esophageal cancer, bile duct cancer, and brain cancer.

In one or more embodiments, the senescence-associated secretory phenotype is a senescence-associated secretory phenotype caused by DNA damage.

In one or more embodiments, the DNA damage is a DNA damage induced by a chemotherapeutic drug.

In one or more embodiments, the chemotherapeutic drug is a genotoxic drug; more preferably, comprising: mitoxantrone, doxorubicin and bleomycin.

In one or more embodiments, the chemotherapeutic drug is mitoxantrone, wherein the weight ratio of mitoxantrone to salvianolic acid E is 1:20˜80; preferably, the weight ratio of mitoxantrone to salvianolic acid E is 1:30˜70; more preferably, the weight ratio of mitoxantrone to salvianolic acid E is 1:40˜60; or the chemotherapeutic drug is bleomycin, with a final concentration of 30˜70 μg/mL, preferably 40˜60 μg/mL, more preferably 45˜55 μg/mL; and the salvianolic acid E has a final concentration of 200˜550 μM, preferably 250˜500 μM, more preferably 300˜420 μM; or
the chemotherapeutic drug is bleomycin, with a final concentration of 30˜70 μg/mL, preferably 40˜60 μg/mL, more preferably 45˜55 μg/mL. and the salvianolic acid E has a final concentration of 700˜5000 μM, preferably 750-4000 μM, more preferably 750-3500 μM; or
the chemotherapeutic drug is doxorubicin, wherein the weight ratio of doxorubicin to salvianolic acid E is 1:4 to 1:16; preferably, the weight ratio of doxorubicin to salvianolic acid E is 1:6 to 1:14; more preferably, the weight ratio of doxorubicin to salvianolic acid E is 1:8 to 1:12.

In the second aspect, the present disclosure provides a method for inhibiting senescence, prolonging lifespan or prolonging the survival period of the old or inhibiting a senescence-associated secretory phenotype, comprising administering to a subject in need of treatment with salvianolic acid E or a pharmaceutically acceptable salt, ester, isomer, solvate or prodrug thereof.

The present disclosure also provides a use of salvianolic acid E or a pharmaceutically acceptable salt, ester, isomer, solvate or prodrug thereof, for:
preparing a composition for inhibiting senescence; or
preparing a composition for prolonging lifespan or prolonging the survival period of the old; or
preparing a composition for specifically-targeted clearance of senescent cells in a tumor microenvironment, or
preparing a composition for inhibiting the senescence-associated secretory phenotype; preferably, the salvianolic acid E specifically targets and induces the senescent cells in the tumor microenvironment to enter the death program.

In the third aspect, the present disclosure provides a use of salvianolic acid E or a pharmaceutically acceptable salt, ester, isomer, solvate or prodrug thereof, in the manufacture of a medicament or preparation, and the medicament or preparation is used for: downregulating a senescence-associated secretory phenotype (SASP), reducing expression or activity of a SASP factor, reducing expression or activity of a cell senescence marker factor, inducing apoptosis of a non-proliferating cell, reducing or eliminating a non-proliferating cell, delaying senescence, prolonging lifespan of a subject, reducing an age-related disease burden in a subject, preventing, alleviating and treating a disease benefiting from the reduction or elimination of non-proliferating cells, reducing resistance to a cancer therapy, enhancing efficacy of an agent capable of inducing cell senescence, promoting tumor regression, reducing tumor size, preventing or treating a cancer, or prolonging cancer survival.

In the fourth aspect, the present disclosure provides a composition, comprising: (a) salvianolic acid E or a pharmaceutically acceptable salt, ester, isomer, solvate or prodrug thereof, and (b) a chemotherapeutic drug, with a capacity of inducing a senescence-associated secretory phenotype in the tumor microenvironment after administration.

In one or more embodiments, the chemotherapeutic drug is a genotoxic drug.

In one or more embodiments, the chemotherapeutic drug comprises: mitoxantrone, doxorubicin and bleomycin.

In the fifth aspect, the present disclosure provides a pharmaceutical composition, comprising: (a) salvianolic acid E or a pharmaceutically acceptable salt, ester, isomer, solvate or prodrug thereof, and (b) a chemotherapeutic drug, with a capacity of inducing a senescence-associated secretory phenotype in the tumor microenvironment after administration, and an optional pharmaceutically acceptable auxiliary material.

In one or more embodiments, the chemotherapeutic drug is a genotoxic drug.

In one or more embodiments, the chemotherapeutic drug comprises: mitoxantrone, doxorubicin and bleomycin.

In the fifth aspect, the present disclosure provides a pharmaceutical composition, comprising: (a) salvianolic acid E or a pharmaceutically acceptable salt, ester, isomer, solvate or prodrug thereof, and (b) a chemotherapeutic drug, with a capacity of inducing a senescence-associated secretory phenotype in the tumor microenvironment after administration, and an optional pharmaceutically acceptable auxiliary material.

In one or more embodiments, the chemotherapeutic drug is a genotoxic drug.

In one or more embodiments, the chemotherapeutic drug comprises: mitoxantrone, doxorubicin and bleomycin.

In the sixth aspect, the present disclosure provides a drug kit for specifically-targeted clearance of senescent cells in a tumor microenvironment and tumor inhibition, comprising a combination of a salvianolic acid E compound or a pharmaceutically acceptable salt, ester, isomer, solvate or prodrug thereof, and a chemotherapeutic drug; wherein the chemotherapeutic drug is capable of inducing a senescence-associated secretory phenotype in the tumor microenvironment after administration.

In one or more embodiments, the chemotherapeutic drug is a genotoxic drug.

In one or more embodiments, the chemotherapeutic drug comprises: mitoxantrone, doxorubicin and bleomycin.

In the seventh aspect, the present disclosure provides a method for preparing a composition, a pharmaceutical composition or a drug kit for inhibiting tumor, comprising mixing the salvianolic acid E compound or a pharmaceutically acceptable salt, ester, isomer, solvate or prodrug thereof and the chemotherapeutic drug; or placing the salvianolic acid E compound or a pharmaceutically acceptable salt, ester, isomer, solvate or prodrug thereof and the chemotherapeutic drug in the same drug kit.

In one or more embodiments, the chemotherapeutic drug is a genotoxic drug.

In one or more embodiments, the chemotherapeutic drug comprises: mitoxantrone, doxorubicin and bleomycin.

In one or more embodiments, the chemotherapeutic drug is mitoxantrone, wherein the weight ratio of mitoxantrone to salvianolic acid E is 1:20˜80; preferably, the weight ratio of mitoxantrone to salvianolic acid E is 1:30˜70; more preferably, the weight ratio of mitoxantrone to salvianolic acid E is 1:40˜60; or
the chemotherapeutic drug is bleomycin, with a final concentration of 30˜70 μg/mL, preferably 40˜60 μg/mL, more preferably 45˜55 μg/ml. and the salvianolic acid E has a final concentration of 200˜550 μM, preferably 250˜500 μM, more preferably 300˜420 μM; or
the chemotherapeutic drug is bleomycin, with a final concentration of 30˜70 μg/mL, preferably 40˜60 μg/mL, more preferably 45˜55 μg/mL. and the salvianolic acid E has a final concentration of 700˜5000 μM, preferably 750-4000 μM, more preferably 750-3500 μM; or
the chemotherapeutic drug is doxorubicin, wherein the weight ratio of doxorubicin to salvianolic acid E is 1:4 to 1:16; preferably, the weight ratio of doxorubicin to salvianolic acid E is 1:6 to 1:14; more preferably, the weight ratio of doxorubicin to salvianolic acid E is 1:8 to 1:12.

In the eighth aspect, the present disclosure provides a method for screening a potential substance for promoting salvianolic acid E to clear senescent cells in a tumor microenvironment, or inhibit tumor, or prolong the lifespan, wherein the method comprises:
(1) providing a system of tumor microenvironment, wherein the system comprises tumor cells and stromal cells;
(2) treating the system of (1) with a chemotherapeutic drug, inducing a senescence-associated secretory phenotype in the tumor microenvironment and administering salvianolic acid E before, during or after inducing the senescence-associated secretory phenotype in the tumor microenvironment;
(3) adding the candidate substance to the system in (2) and observing its effect on the tumor microenvironment system; if the candidate substance statistically promotes salvianolic acid E to clear senescent cells in a tumor microenvironment, then the candidate substance is a potential substance that can be used in combination with salvianolic acid E to clear senescent cells in the tumor microenvironment or inhibit tumors or prolong the lifespan.

In one or more embodiments, cell apoptosis or the status of the senescence-associated secretory phenotype (SASP) can be evaluated by observing caspase-3/7 activity or the expression of SASP factors.

In one or more embodiments, the SASP factors comprise but are not limited to: IL6, CXCL8, SPINK1, WNT16B, GM-CSF, MMP3, CXCL1, CXCL3, IL-1a, IL-1B; or cell apoptosis or the status of the senescence-associated secretory phenotype (SASP) can be evaluated by observing senescence marker p16INK4A in animals administering with a chemotherapeutic drug.

In the ninth aspect, the present disclosure provides a method for screening a potential substance for inhibiting senescence-associated secretory phenotype, wherein the method comprises:
(1) providing a system of stromal cells, inducing the senescence-associated secretory phenotype in the system; administering salvianolic acid E before, during or after inducing the senescence-associated secretory phenotype in the system;
(2) adding the candidate substance to the system of (1) and observing its effect on the system of stromal cells. If the candidate substance statistically promotes salvianolic acid E to inhibit the senescence-associated secretory phenotype, then the candidate substance is a potential substance that can be used in combination with salvianolic acid E to inhibit the senescence-associated secretory phenotype.

Other aspects of the present disclosure will be apparent to those skilled in the art based on the disclosure herein.

### DESCRIPTION OF FIGURES

FIG. 1 shows the results of SA-β-Gal staining of proliferating human stromal cells PSC27 (early passages: such as p10-20) 7-10 days after they were in vitro treated with chemotherapeutic drug bleomycin (BLEO) at a concentration of 50 μg/ml. The upper part shows representative images and lower part displays statistical data. CTRL, control cells; BLEO, cells treated with bleomycin. ***, P<0.001.

FIG. 2 shows the results of BrdU staining of PSC27 cells treated with chemotherapeutic drug bleomycin (BLEO). The upper part shows representative images and lower part presents statistical data. CTRL, control cells; BLEO, cells treated with bleomycin. ****, P<0.0001.

FIG. 3 shows the results of immunofluorescence staining using γH2AX after PSC27 cells were treated with chemotherapeutic drug bleomycin (BLEO). CTRL, control cells; BLEO, cells treated with bleomycin. ***, P<0.001. According to the number of fluorescent spots in the nucleus, they were divided into 4 categories, including single cells with 0 foci, 1 to 3 foci, 4 to 10 foci, and >10 foci.

FIG. 4 shows an experimental flow chart for screening organic chemical drug libraries to obtain small molecules (including natural and synthetic compounds) with anti-senescence activity.

FIG. 5 shows a schematic diagram of the chemical structure of SAE.

FIG. 6 shows that after the RNA-seq data analysis, the heatmap showed that the expression of a large number of factors in senescent cells caused by BLEO damage is upregulated, but many of them are significantly reversed after SAE treatment. Red star mark, typical SASP exocrine factor.

FIG. 7 shows that in the results of GSEA analysis, the expressions of SASP specific molecular marker or related factors are intensively upregulated in BLEO-induced senescent cells, but significantly decreased after SAE treatment of senescent cells.

FIG. 8 shows that in the results of GSEA analysis, the expressions of NF-κB molecular marker or related factors are intensively upregulated in BLEO-induced senescent cells, but significantly decreased after SAE treatment of senescent cells.

FIG. 9 shows the representative pathways of biological process for 100 molecules with significant down-regulation caused by SAE in senescent cells according to the KEGG pathway analysis.

FIG. 10 shows the representative pathways of cellular component for 100 molecules with significant down-regulation caused by SAE in senescent cells according to the KEGG pathway analysis.

FIG. 11 shows the relative expression levels of a group of typical SASP molecules in senescent cells induced by BLEO and treated with different concentrations of SAE according to the detection and analysis of fluorescent quantitative PCR (qRT-PCR). All data are normalized results compared to the CTRL group. *P<0.05, **P<0.01, ***P<0.001.

FIG. 12 shows whether PSC27 is senescent or not according to the determination by SA-β-Gal staining under the condition of increasing SAE concentration. \hat{ }, P>0.05; **, P<0.01; ****, P<0.0001. The P value of positive proportion of cells in these experimental groups for SAE at the concentrations of 100 μM, 200 μM, 400 μM, 800 μM, 1600 μM, 2000 μM and 3000 μM are statistically significant compared with the data at 0 μM.

FIG. 13 shows the representative pictures of PSC27 under various conditions after SA-β-Gal staining. 3 repetitions per group, arranged up and down. Scale bar, 30 μm.

FIG. 14 shows that CCK8 detects the survival rates of proliferating cells and cells in the senescent group under increasing concentrations of SAE. P value at each SAE concentration shows significant difference after comparison between CTRL and BLEO groups. **, P<0.01; ***, P<0.001; ****, P<0.0001.

FIG. 15 shows the population doubling (PD) test of PSC27. The cells were damaged by BLEO at passage 10 (p10), and then SAE was added to the medium at day 8. The effect SAE on cell proliferation potential is determined by comparative analysis of the population doubling (PD) of CTRL group, BLEO group, SAE group and BLEO/SAE group. \hat{ }, P>0.05; ***P<0.001.

FIG. 16 shows that the caspase 3/7 activity was induced during the SAE treatment of senescent cells. PSC27 cells gradually entered the senescent stage after being treated with BLEO for 12 hours under culture conditions. 200 μM SAE was added to the medium of senescent cells starting from day 7, NucLight Rapid Red reagent was used to label the cells and caspase 3/7 reagent (IncuCyte) was used for apoptosis detection. Caspase 3/7 activity was detected every 4 hours (n=3).

FIG. 17 shows the senolytic activity reversed by pan-caspase inhibitor (20 μM QVD-OPh) (200 μM SAE was used in this experiment, and 1.25 μM ABT263 was used as a positive control; the latter is a senescent cell apoptosis inducer reported in recent years). Statistical differences are obtained by two-way ANOVA (Turkey'test).

FIG. 18 shows the apoptosis of PSC27 under several conditions measured by flow cytometry. Q2, distribution area of early apoptotic cells; Q3, distribution area of late apoptotic cells.

FIG. 19 shows the comparative analysis of the numbers of survival and apoptotic cells treated with BLEO and/or SAE. ***, P<0.001; ****, P<0.0001.

FIG. 20 shows a schematic diagram of the administration of mice in the preclinical trial. Human stromal cells PSC27 and cancer cells PC3 were mixed in vitro (1:4) and then transplanted into mice subcutaneously to form transplanted tumors. After multiple treatment cycles under the condition of single drug or combined drug administration, the mice were finally sacrificed, and the expression changes of relevant molecules in tumor tissues were analyzed pathologically.

FIG. 21 shows that PC3 mixed with PSC27 cells of the CTRL group or BLEO injury group, or PC3 cells alone were transplanted into the subcutaneous tissue of mice to form transplanted tumors. At the end of the 8th week, the mice were dissected and the tumors were obtained, with the volumes of the tumors under the conditions of each group detected and compared. **, P<0.01; ***, P<0.001; ****, P<0.0001.

FIG. 22 shows a schematic diagram of the administration time and administration method for the mice in the preclinical trial. Every two weeks was a dosing cycle, and MIT (mitoxantrone) was intraperitoneally administered to the mice on the first day of the 3rd/5th/7th weeks. From the first day of the 5th week, the mice were intraperitoneally administered with SAE, once a week. After the 8-week course of treatment, the mice were dissected for pathological identification and expression analysis.

FIG. 23 shows the tumor terminal volume statistical analysis. Chemotherapeutic drug MIT alone or together with anti-senescence drug SAE was used to administer mice, and after the 8th week, the tumor size of each group was compared and analyzed.

FIG. 24 shows the comparison of cell senescence in PC3/PSC27 tumor-bearing animal lesions in preclinical experiments. Representative pictures after SA-β-Gal staining. Scale bar, 100 μm.

FIG. 25 shows the parallel analysis of the percentage of positive cells stained by SA-β-Gal in tumor tissues in mice. \hat{ }, P>0.05; *, P<0.05; ****, P<0.0001.

FIG. 26 shows the expression of SASP typical factors in epithelial cancer cells and stromal cells in mice lesions detected and analyzed by fluorescence quantitative PCR (qRT-PCR). The stromal cells and cancer cells were specifically separated by LCM technology, total RNA was prepared and used for SASP expression detection. \hat{ }, P>0.05; *, P<0.05; **, P<0.01.

FIG. 27 shows the expression of SASP factors in the stromal cells in mice lesions administered with vehicle, MIT and MIT/SAE detected and analyzed by fluorescence quantitative PCR (qRT-PCR). *, P<0.05; **, P<0.01; ***, P<0.001.

FIG. 28 shows that the ratio of DNA damage and apoptosis in mice of each group was analyzed after specific separation of cancer cells in the lesion by LCM technology. \hat{ }, P>0.05; *, P<0.05; **, P<0.01.

FIG. 29 shows the comparison of Kaplan Meier data of disease-free survival in NOD/SCID mice after various drug treatments. When the tumor volume in the vehicle, MIT, SAE and MIT/SAE groups exceeds 2000 mm3, it is considered that severe disease has occurred, and the mice need to be killed in time and the tumor bearing status should be detected. \hat{ }, P>0.05; **, P<0.01.

FIG. 30 shows the comparative analysis of the mice body weight data at the end of the course of treatment under various administration conditions. \hat{ }, P>0.05.

FIG. 31 shows the comparative analysis of the mice serological data at the end of the course of treatment under the above various administration conditions. Creatinine, urea (kidney indicator), ALP and ALT (liver indicator) data were compared in parallel. \hat{ }, P>0.05.

FIG. 32 shows the comparative analysis of body weight data of immune intact mice (C57BL/6J) at the end of the course of treatment under various administration conditions. \hat{ }, P>0.05.

FIG. 33 shows the comparative analysis of mouse blood cell counts at the end of the course of treatment under various administration conditions in the preclinical trial. WBC, lymphocytes and neutrophils were compared in parallel. \hat{ }, P>0.05.

FIG. 34 shows the tumor terminal volume statistical analysis. Chemotherapeutic drug DOX alone or together with anti-senescence drug SAE was administered to mice, and the tumor size of each group was compared and analyzed after the end of the 8th week.

FIG. 35 shows the tumor terminal volume statistical analysis. Chemotherapeutic drug BLEO alone or together with anti-senescence drug SAE was used to administer mice, and after the 8th week, the tumor size of each group was compared and analyzed.

FIG. 36 shows the tumor terminal volume statistical analysis. Chemotherapeutic drug DOC alone or together with anti-senescence drug SAE was administered to mice, and the tumor size of each group was compared and analyzed after the end of the 8th week.

FIG. 37 shows the tumor terminal volume statistical analysis. Chemotherapeutic drug VIN alone or together with anti-senescence drug SAE was administered to mice, and the tumor size of each group was compared and analyzed after the end of the 8th week.

FIG. 38 shows the tumor terminal volume statistical analysis. Chemotherapeutic drug MIT alone or together with another molecule SAI of salvianolic acid family was used to administer mice, and after the 8th week, the tumor size of each group was compared and analyzed.

FIG. 39 shows the tumor terminal volume statistical analysis. Chemotherapeutic drug MIT alone or together with another molecule SAJ of salvianolic acid family was used to administer mice, and after the 8th week, the tumor size of each group was compared and analyzed.

FIG. 40 shows the tumor terminal volume statistical analysis. Chemotherapeutic drug MIT alone or together with salvianolic acid extracts (SAE) or green tea extracts (GTE) was used to administer mice, and after the 8th week, the tumor size of each group was compared and analyzed.

FIG. 41 shows the comprehensive comparison and comprehensive analysis of the effects of extracts or small molecule compounds from different natural plant sources on reducing tumor volume when combined with the chemotherapeutic drug MIT to treat tumor-bearing mice.

FIG. 42 shows the post-treatment survival curves for mice in preclinical stage. Starting at 24 to 27 months of age, C57BL/6 mice received intraperitoneal administration of vehicle or SAE every two weeks (n=80 for vehicle group; n=91 for SAE group). The median survival of animals in each group was calculated and indicated. ****, P<0.0001.

FIG. 43 shows the overall (lifetime, or full length) survival curves for mice in preclinical stage. Starting at 24 to 27 months of age, C57BL/6 mice received intraperitoneal administration of vehicle or SAE every two weeks (n=80 for vehicle group; n=91 for SAE group). The median lifetime survival of animals in each group was calculated and indicated. ****, P<0.0001.

### DETAILED DESCRIPTION OF THE INVENTION

After in-depth investigations in screening drugs targeting tumor microenvironment and eliminating senescent cells, the inventors revealed that salvianolic acid E exerts its effects by targeting tumor microenvironment and the removal of senescent cells. When combined with chemotherapeutic drugs, it shows extremely significant effects in promoting tumor suppression by removing senescent stromal cells. For a senescence-associated secretory phenotype (SASP), the SAE can also effectively target senescent cells so as to inhibit the SASP. Besides, the SAE can also significantly prolong the lifespan of animals, significantly prolong the survival period of the old, and improve the life quality of animals.

The inventors have found that, although SAE can specifically target and eliminate senescent cells in the tumor microenvironment, it shows no specific inhibitory effect on tumor cells. Chemotherapeutic drugs are capable of inhibiting tumor cells, also with a significant effect on the tumor microenvironment. However, chemotherapeutic drugs may lead to notable side effects, particularly the formation and development of SASP, and prolonged use of chemotherapy drugs also tends to induce drug resistance in tumor cells. Surprisingly, the combined use of SAE with certain specific chemotherapeutic drugs can effectively exhibit a benign complementary effect against the disease, achieving unexpectedly enhanced results.

### Salvianolic Acid E (Salvianolic Acid, SAE)

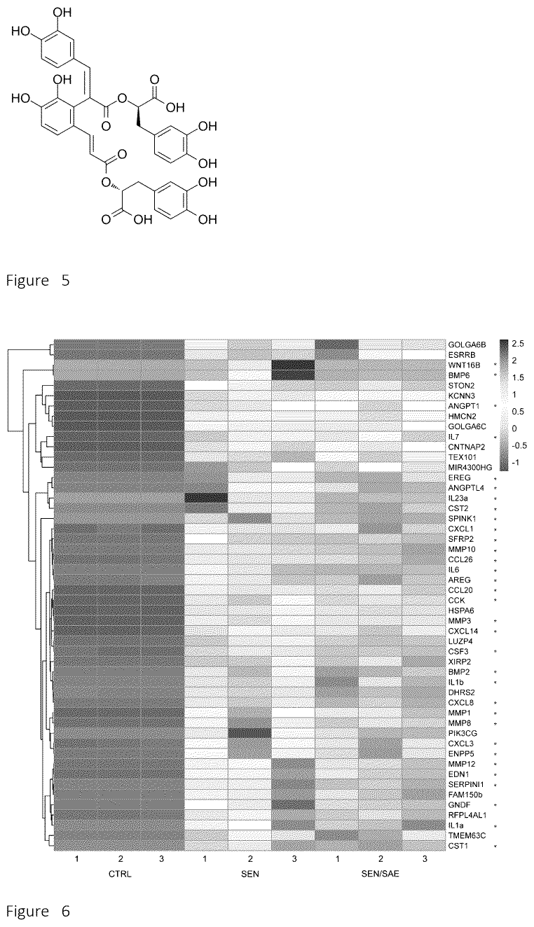

As used herein, “Salvianolic acid E”, also referred to as “salvianolic acid E”, “Salvianolic acid E”, “SalE” or “SAE”, is a monomeric compound extracted from the traditional Chinese herb Danshen. The molecular formula of salvianolic acid E is C36H30016, and its CAS number is 142998-46-7. Salvianolic acid E is present in Danshen at a very low concentration, and there are almost no reported studies on its pharmacological effects. The chemical structure is shown in FIG. 5. In the present disclosure, “compound” (including salvianolic acid E, salt or prodrug thereof, etc.) can be a compound in pure form, or a compound with a purity greater than 85% (preferably greater than 90%, such as 95%, 98%, 99%).

Those skilled in the art should understand that, after knowing the structure of the compound of the present disclosure, the compound of the present disclosure can be obtained by various methods well known in the art, by using known raw materials in the art, such as methods of chemical synthesis or extraction from organisms (e.g., microorganisms), these methods are all included in the present disclosure. In addition, the salvianolic acid E is also a commercial drug, so a finished product thereof is easily available to those skilled in the art.

In the present disclosure also comprises a pharmaceutically acceptable salt, ester, isomer, solvate or prodrug of the salvianolic acid E, as long as they retain the same or substantially same functions with the salvianolic acid E compound. In the present disclosure, a “pharmaceutically acceptable” component is one that is suitable for use with humans and/or animals without undue adverse side effects (such as toxicity, irritation, and allergic response) commensurate with a reasonable benefit risk ratio. The “pharmaceutically acceptable salt” can be an acid salt or basic salt of the salvianolic acid E. “Pharmaceutically acceptable acid salt” refers to a salt that can maintain the biological activity and properties of the free base, and such salt will not have undesired biological activity or other changes. Such salts may be formed from inorganic acids such as, but not limited to, hydrochloric acid, hydrobromic acid, sulfuric acid, nitric acid, phosphoric acid, and the like. Such salt may also be formed from an organic acid, for example, but not limited to, acetic acid, dichloroacetic acid, adipic acid, alginic acid, ascorbic acid, aspartic acid, benzenesulfonic acid, benzoic acid, 4-acetamidobenzoic acid, camphoric acid, camphorsulfonic acid, capric acid, caproic acid, caprylic acid, carbonic acid, cinnamic acid, citric acid, cyclamic acid, dodecylsulfonic acid, 1,2-ethanedisulfonic acid, ethanesulfonic acid, isethionic acid, formic acid, fumaric acid, galactaric acid, gentisic acid, glucoheptonic acid, gluconic acid, glucuronic acid, glutamic acid, glutaric acid, 2-oxoglutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, 2-naphthale-nesulfonic acid, 1-naphthol-2-carboxylic acid, niacin, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-tolue-nesulfonic acid, trifluoroacetic acid, undecylenic acid and the like.

“Pharmaceutically acceptable basic salt” refers to a salt that can maintain the biological activity and properties of the free acid, and such salts will not have undesired biological activity or other changes. These salts are prepared by adding an inorganic or organic base to the free acid. Salts derived from inorganic bases include, but are not limited to, slats of sodium, potassium, lithium, ammonium, calcium, magnesium, iron, zinc, copper, manganese, aluminum and the like. Preferred inorganic salts are slats of ammonium, sodium, potassium, calcium and magnesium. Salts derived from organic bases include, but are not limited to, primary, secondary, and tertiary ammonium salts. Substituted amines include naturally substituted amines, cyclic amines, and basic ion exchange resins, such as ammonia, isopropylamine, trimethylamine, diethylamine, triethylamine, tripropylamine, diethanolamine, ethanolamine, tannol, 2-dimethylaminoethanol, 2-diethylaminoethanol, dicyclohexylamine, lysine, arginine, histidine, caffeine, procaine, halamine, choline, betaine, phenethylbenzylamine, N,N′-bisbenzylethylenediamine, ethylenediamine, glucosamine, alphaglucosamine, cocaine, theobromine, triamine ethanolamine, ceramide, purine, piperazine, piperidine, N-ethylpiperidine, polyamide resin and the like. Preferred organic bases are isopropylamine, diethylamine, ethanolamine, trimethylamine, dicyclohexylamine, choline and caffeine.

The compound disclosed in the present disclosure may exist as a solvate (such as a hydrate), including monohydrate, dihydrate, hemihydrate, sesquihydrate, trihydrate, tetrahydrate, and similar structures. In the present disclosure, a prodrug of the salvianolic acid E is also included, and the “prodrug” refers to a compound that undergoes metabolism or chemical reaction in the body of a subject and converts into the desired salvianolic acid E after being taken in an appropriate way. In the present disclosure, isomers of salvianolic acid E are also included. This is because compounds have one or more asymmetric centers, so these compounds can exist as racemates, individual enantiomers, individual diastereomers, mixtures of diastereomers, cis isomers or trans isomers, and the like. Those skilled in the art should understand that, after knowing the structure of the compound of the present disclosure, the compound of the present disclosure can be obtained by various methods well known in the art, by using known raw materials in the art, such as methods of chemical synthesis or extraction from organisms (e.g., animals or plants), these methods are all included in the present disclosure. The compounds of the present disclosure can be synthesized by methods known in the art. The synthesized compounds can be further purified by methods such as column chromatography, high-performance liquid chromatography (HPLC), and so on. In addition, compounds of the present disclosure are commercially available.

The inventors found that salvianolic acid E can effectively inhibit the expression of SASP and significantly reduce the survival rate of senescent cells.

Therefore, the present disclosure provides a use of salvianolic acid E in the manufacture of a medicament or preparation, wherein the medicament or preparation is used for: downregulating a senescence-associated secretory phenotype (SASP), reducing expression or activity of a SASP factor, reducing expression or activity of a cell senescence marker factor, inducing apoptosis of a non-proliferating cell, reducing or eliminating a non-proliferating cell, delaying senescence, prolonging lifespan of a subject, reducing an age-related disease burden in a subject, preventing, alleviating and treating a disease benefiting from the reduction or elimination of non-proliferating cells, reducing resistance to a cancer therapy, enhancing efficacy of an agent capable of inducing cell senescence, promoting tumor regression, reducing tumor size, preventing or treating a cancer, or prolonging cancer survival. Herein, a subject or a patient generally refers to a mammal, especially a human.

In this article, “elimination” and “clearance” are used interchangeably, indicating that the substance uses the cell's own mechanism to selectively destroy non-proliferating cells (senescent cells) to achieve the effect of cell death and clearance. In an exemplary embodiment, a substance (e.g., SAE) can eliminate or clean non-proliferating cells by inducing apoptosis.

“SASP factors” as used herein include extracellular matrix proteins, inflammatory cytokines and cancer cell growth factors. The SASP factors may include the factors shown in FIG. 26 or one or more selected from: IL6, CXCL8, MCP2, CXCL1, GM-CSF, MMP3, AREG, SFRP2, ANGPTL4, IL1a.

The “disease benefiting from the reduction or elimination of non-proliferating cells” described herein are generally an age-related disease, including but not limited to cancer, cardiovascular and cerebrovascular disease, osteoporosis, age-related degenerative joint disease (e.g., arthritis), metabolic disease, neurodegenerative disease. Preferably, the cancer is a prostate cancer.

Herein, salvianolic acid E (SAE) can also be used to extend the lifespan of a subject and reduce age-related disease burden in a subject. In some embodiments, the subject is an elderly subject, for example, the subject corresponding to a mouse of at least 20 months of age or a human of at least 60 years of age. Preferably, the elderly subject is a subject corresponding to a mouse of at least 24 months of age or a human of at least 75 years of age. More preferably, the elderly subject is a subject corresponding to a mouse of 24-27 months of age or a human of 75-90 years of age. Although the elderly subject is used as a research subject in specific embodiments, this is only an example for the convenience of result analysis (for example, an older subject has more age-related diseases). Based on the efficacy of salvianolic acid E in eliminating senescent cells found in the present disclosure, those skilled in the art should know that it can be used in a subject of any age to eliminate senescent cells, prolong lifespan and reduce the burden of an age-related disease.

Herein, the salvianolic acid E (SAE) can also be used to reduce resistance to cancer therapy in a patient. The cancer therapy includes chemotherapy or radiation therapy; examples of chemotherapy include cytotoxic therapy such as MIT or DOX, and examples of radiation therapy include ionizing radiation, mainly including therapies with α-rays, β-rays, y-rays and X-rays as well as proton and neutron flows.

Furthermore, the inventors found that when used in combination with a certain agent, the salvianolic acid E (SAE) can enhance the cytotoxicity of the agent for inducing cell senescence. The agent for inducing cell senescence may be an agent that induces to produce senescent cells by causing DNA damage and/or apoptosis, and examples thereof include chemotherapeutic agents or radiation.

Therefore, the present disclosure also provides a use of the salvianolic acid E in enhancing the efficacy of an agent that induces cell senescence, and a use of salvianolic acid E in combination with an agent that induces cell senescence in promoting tumor regression, reducing tumor volume, preventing or treating cancer, and prolonging cancer survival.

Exemplarily, the cell is a tumor cell; the tumor is a prostate tumor; and the cancer is a prostate cancer.

In another aspect of the present disclosure, there is provided a method for achieving the above use, and the method comprises: using (a) the salvianolic acid E described herein or pharmaceutically acceptable salt, hydrate or prodrug thereof, and optionally (b) an agent capable of inducing a senescent cell in a subject, to treat a senescent cell or administer them to a subject in need thereof. The term “administering” or “giving” as used herein refers to providing a compound or pharmaceutical composition of the present disclosure to a subject suffering from a disease or condition to be treated or prevented or at risk of a disease or condition.

### Compositions, Pharmaceutical Compositions of Salvianolic Acid E (SAE)

The composition (for example, pharmaceutical composition) of the present disclosure uses a substance such as E or a salt, ester, isomer, solvate or prodrug thereof as an active component. As mentioned above, the composition containing the E (e.g., SAE) is capable of down-regulating senescence-associated secretory phenotype (SASP), reducing expression or activity of SASP factors, reducing expression or activity of cell senescence marker factors, inducing apoptosis of non-proliferating cells (senescent cells), reducing or eliminating non-proliferating cells (senescent cells), delaying aging, prolonging lifespan in a subject, reducing age-related disease burden in a subject, preventing, relieving and treating a diseases that would benefit from the reduction or elimination of non-proliferating cells, reducing resistance to cancer therapy. In the present disclosure, the “comprising”, “having” or “including” includes the terms “containing”, “mainly consisting of” and “essentially consisting of”, and “consisting of”; “mainly composed of”, “basically composed of” and “consisting of” belong to the subordinate concepts of “comprising”, “having” or “including”.

When the composition further comprises a genotoxic drug (e.g., a chemotherapeutic agent) as an active component, the composition can promote tumor regression, reduce tumor volume, prevent or treat a cancer, and prolong cancer survival.

When the composition described herein is used as a medication, it also comprises a pharmaceutically acceptable auxiliary material. “Pharmaceutically acceptable auxiliary material” is a pharmaceutically or food-acceptable carrier, solvent, suspending agent or excipient that can be used to deliver the active components in the composition of the present disclosure (e.g., E and an optional genotoxic drug) to an animal or human. Exemplary auxiliary material can be a liquid or solid, and includes, but is not limited to: pH adjuster, surfactant, carbohydrate, adjuvant, antioxidant, chelating agent, ionic strength enhancer, preservative, carrier, glidant, sweetener, dye/coloring agent, flavor enhancer, wetting agent, dispersant, suspending agent, stabilizer, isotonic agent, solvent, emulsifier, atomizing agent, compressed air or other suitable gases, or other suitable inactive ingredients used in combination with medicinal compounds. More specifically, suitable auxiliary material may be those commonly used in the art for the administration of small molecule compounds. Examples of auxiliary material include various lactoses, mannitols, oils such as corn oil, buffers such as PBS, saline, polyethylene glycol, glycerol, polypropylene glycol, dimethylsulfoxide, amides such as dimethylacetamide, proteins such as white protein, and detergents such as Tween 80, monosaccharides and oligopolysaccharides such as glucose, lactose, cyclodextrin and starch. In general, the composition will contain a therapeutically effective amount of the active ingredient described herein. A therapeutically effective amount refers to a dose that can achieve treatment, prevention, alleviation and/or alleviation of a disease or condition in a subject. The effective amount of the E in the present disclosure may vary with the mode of administration, the severity of the disease to be treated, and the like. Selection of preferred effective amount can be determined by those skilled in the art based on various factors (e.g. through clinical trials). Such factors comprise but are not limited to: pharmacokinetic parameters (such as bioavailability, metabolism, half-life, and so on) of the SAE, the severity of the disease to be treated, weight, immune status of patients, the form of administration, and so on. The therapeutically effective dose can be determined according to factors such as the patient's age, sex, disease and its severity, and other physical conditions of the patient. A therapeutically effective amount may be administered as a single dose, or may be administered in multiple doses in accordance with an effective treatment regimen. Herein, a subject or a patient generally refers to a mammal, especially a human. Exemplarily, the composition comprises, for example, 0.001-50%, preferably 0.01-30%, more preferably 0.05-10% by weight of the active ingredients (e.g., the E and an optional genotoxic drug).

Under specific conditions, usage frequency of senolytic drugs may depend on the accumulation rate of senescent cells. However, the accumulation rate of senescent cells may vary depending on the environment in which cellular senescence occurs. For example, repeated exposure to DNA-damaging cancer therapies or sustained high-fat diets may lead to a more rapid re-accumulation of senescent cells compared to natural senescence. Intermittent use of senolytics can reduce the risk of adverse reactions in patients and allow for the use of senolytics during healthy periods. In addition, intermittent administration can reduce the side effects of senolytics and reduce the likelihood of patients developing drug resistance. Contrary to the situation with anticancer drugs or antibiotics, because senescent cells do not undergo division, the body cannot rely on cell proliferation to generate senolytics resistance and thus cannot acquire favorable mutations. This creates a favorable foundation for the widespread clinical use of senolytics.

The pharmaceutical composition or mixture of the present disclosure can be prepared into any conventional preparation form by conventional methods. Dosage forms can be diverse, the dosage form is acceptable as long as the active ingredients can effectively reach the body of the mammal. For example, it can be selected from: injection, infusion, tablet, capsule, pill. Wherein, the active components (e.g., the E and an optional genotoxic drug) can exist in a suitable solid or liquid carrier or diluent. The mixture of active ingredients or the pharmaceutical composition of the present disclosure may also be stored in a sterile device suitable for injection or infusion. The effective doses of the active ingredients in the composition (e.g., the E and an optional genotoxic drug) may vary with the administration mode and the severity of the disease to be treated, which can be based on the experience and recommendations of clinicians.

In a specific embodiment of the present disclosure, there is provided a series of dosage regimens for the E and the optional genotoxic drug according to different molar ratios or mass ratios. In the present disclosure, mice are also used as experimental animals. It is easy for those skilled in the art to convert the dosage for mice into the dosage suitable for humans. For example, it can be calculated according to the Meeh-Rubner formula:

A=k×(W2/3)/10000.

In the formula, A is the body surface area, calculated in m2; W is the body weight, calculated in g; K is a constant, which varies with animal species. Generally speaking, mice and rats are 9.1, guinea pigs are 9.8, rabbits are 10.1, cats are 9.9, dogs are 11.2, monkeys are 11.8, human is 10.6. It will be understood that, depending on the drug and the clinical situation, the conversion of the administered dose may vary according to the assessment of an experienced pharmacist.

The E and an optional genotoxic drug or the pharmaceutical composition can be administered orally, intravenously, intramuscularly or subcutaneously, etc. Oral administration may be preferred. Pharmaceutical forms suitable for oral administration include, but are not limited to, tablet, powder, capsule, sustained-release formulation, and the like. The pharmaceutical forms suitable for injection include: sterile aqueous solution or dispersion and sterile powder. In all cases, these forms must be sterile and must be fluid to easily drain from syringe. When necessary, the E and the optional genotoxic drug can also be administered in combination with an additional active ingredient or drug.

The present disclosure also provides a drug kit or kit for down-regulating or clearing senescent cells, or prolonging lifespan of an organism, and the drug kit or kit comprises the pharmaceutical composition described in any embodiment herein. Alternatively, the drug kit or kit comprises a mixture of the E described herein and the optional genotoxic drug. Alternatively, the drug kit or the kit comprises: Container 1, and the E or a pharmaceutically acceptable salt, hydrate or prodrug thereof described herein placed in Container 1; and Container 2, and the genotoxic drug placed in Container 2.

The drug kit or the kit may also comprise some auxiliary materials, such as measuring tool and container such as syringe and the like required for using or administering the composition in various dosage forms. The drug kit or the kit can also comprise instructions for use, explaining the method of treating, down-regulation or clearing senescent cells or prolonging the survival period of body.

### Use of Salvianolic Acid E (SAE) and Combined Use of SAE and Other Chemotherapeutic Drugs

As previously mentioned, the inventors have discovered that the combined use of salvianolic acid E (SAE) with certain specific chemotherapeutic drugs can effectively exhibit a benign complementary effect targeted to the disease, resulting in remarkably significant synergistic effects.

In the screening of drugs that inhibit SASP expression, the inventors found that although SASP factors are generally significantly upregulated in senescent cells, the expression of SASP factors in senescent cells after SAE treatment is universally reduced, and this effect is highly noticeable.

In the inventors' research, it was also discovered that SAE has an ideal cytotoxic effect on senescent cells at appropriate concentrations. For example, in some embodiments, the inventors found that when SAE reaches a threshold at 2000 M, senescent cells at this concentration remain at 20% or lower. Therefore, at a certain concentration, SAE is a novel senolytic with excellent efficacy and high target specificity.

The inventors also found in the researches that after treatment with genotoxic drugs (such as MIT), there was a significant increase in population doubling (PD) capacity in stromal cells of the combined treatment group of genotoxic drugs and SAE, compared to the cells that rapidly entered a growth arrest state after damaging treatment. After genotoxic treatment, there was a significant increase in population doubling (PD) capacity in stromal cells of the combined treatment group of genotoxic drugs and SAE, compared to the cells that rapidly entered a growth arrest state after damaging treatment. The combination of SAE and genotoxic drugs can rapidly recover the proliferation potential of stromal cells in the short term, leading to a sharp contrast to the single use of genotoxic drugs, which is surprising. SAE itself does not affect PD in proliferating cells, and this data further demonstrates that SAE is selective and target-specific between senescent cells and normal cells. The inventors also found in the researches that after tumors were transplanted into animals, the volume of xenografts composed of PC3 cells and senescent PSC27 cells was significantly increased than that of transplanted tumors composed of PC3 cancer cells and primary PSC27 stromal cells. Compared with the treatment group treated with MIT alone, administration of SAE combined with MIT can significantly reduce tumor size; compared with MIT, tumor volume was reduced by 55.1%; compared with placebo treatment, tumor volume was reduced by 74.6%. This suppressive effect was surprising.

The inventors also found that the MIT administration induced the appearance of a large number of senescent cells in tumor tissues. However, SAE administration essentially depleted most senescent cells in the lesions of these animals treated with chemotherapeutic drugs. After MIT administration, the expression of SASP factors was significantly increased (mainly in stromal cells); however, this change was largely reversed when administered with SAE. When MIT-treated animals are used together with SAE, the index of DNA damage or apoptosis is significantly enhanced, which means that the cytotoxicity of tumor sites in animals under these senolytic drug-treated conditions is enhanced; when SAE is used therapeutically, the activity of caspase 3/4, a typical marker of cell apoptosis, was significantly increased. At the same time, mice treated with the MIT/SAE combination showed the longest median survival; survival was greatly extended. It can be seen that SAE therapeutic targeting of senescent cells can promote tumor suppression and reduce drug resistance of chemotherapy. In the researches of the present inventors, it was also found that under the treatment regimen of mice taking the drug once every two weeks, the SAE group started to be administered from the age of 24 to 27 months (equivalent to the age of 75 to 90 years in humans). The median survival period was 72.8% longer than that of the Vehicle group, with a lower risk of death, indicating that SAE-mediated senescent cell clearance can reduce the risk of death in elderly mice and effectively extend their survival. Intermittent provision of SAE, a bioactive anti-senescence drug, can significantly reduce the burden of the disease in senescent body by clearing senescent cells in the microenvironment, and can increase the lifespan of the body after treatment. This treatment does not lead to a significant increase in the body's morbidity and could in reality be used safely in later stages of life.

Based on new findings of the inventors, the present disclosure provides a use of SAE for preparing a composition for specifically-targeted clearance of senescent cells in a tumor microenvironment or inhibiting tumor; or preparing a composition for inhibiting senescence-associated secretory phenotype.

As used in the present disclosure, unless otherwise specified, the “tumor” is a tumor that exhibits a senescence-associated secretory phenotype in the tumor microenvironment after treatment with genotoxic drugs, and/or is a tumor that develops drug resistance after using genotoxic drugs. Preferably, it comprises: prostate cancer, breast cancer, lung cancer, colorectal cancer, gastric cancer, liver cancer, pancreatic cancer, bladder cancer, skin cancer, kidney cancer, esophageal cancer, bile duct cancer and brain cancer.

As used in the present disclosure, unless otherwise specified, the “chemotherapeutic drug” is a chemotherapeutic drug induced senescence-associated secretory phenotype (SASP) after administration.

In some embodiments of the present disclosure, the “senescence-associated secretory phenotype” is a senescence-associated secretory phenotype causing by DNA damage; preferably, the DNA damage is a DNA damage caused by a chemotherapeutic drug; more preferably, the chemotherapeutic drug comprises a genotoxic drug.

### Drug Screening

After knowing the close correlation and mechanism of SAE and the tumor microenvironment or SASP, this characteristic can be used for screening drugs with further optimized inhibitory effects. Truly useful drugs can be identified from the substances targeting senescent cells in the tumor microenvironment, inhibiting tumors, reversing drug resistance in tumors, or inhibiting/delaying the senescence-associated secretory phenotype. Alternatively, substances from the compounds can also be identified with synergistic effects when combined with SAE. Therefore, the present disclosure provides a method for screening a potential substance for inhibiting senescence-associated secretory phenotype, wherein the method comprises: (1) providing a system of tumor microenvironment, wherein the system comprises tumor cells and stromal cells; (2) treating the system of (1) with a chemotherapeutic drug, inducing a senescence-associated secretory phenotype in the tumor microenvironment; (3) adding the candidate substance to the system in (2) and observing its effect on the tumor microenvironment system; if the candidate substance specifically targets to clear senescent cells and/or promotes the growth of stromal cells (non-senescent cells) in a tumor microenvironment (increase the PD rate of stromal cells), then the candidate substance is a potential substance that can be used for promoting chemotherapeutic drugs to inhibit tumors. In a more preferred embodiment, in step (2), it also comprises: administering salvianolic acid E before, during or after inducing the senescence-associated secretory phenotype in the tumor microenvironment; in step (2), it also comprises: if the candidate substance statistically promotes salvianolic acid E to clear senescent cells and/or promotes the growth of stromal cells in a tumor microenvironment, then the candidate substance is a potential substance that can be used in combination with salvianolic acid E to inhibit tumors.

The present disclosure also provides a method for screening a potential substance for inhibiting senescence-associated secretory phenotype, wherein the method comprises: (1) providing a system of stromal cells, inducing the senescence-associated secretory phenotype in the system; (2) adding the candidate substance to the system of (1) and observing its effect on the system of stromal cells. If the candidate substance statistically promotes salvianolic acid E to inhibit the senescence-associated secretory phenotype, then the candidate substance is a potential substance that can be used in combination with SAE to inhibit the senescence-associated secretory phenotype.

In a preferred embodiment of the present disclosure, when performing screening, a control group may be established to facilitate the observation of indicator changes in the testing group. The control group can be a system without adding the candidate substance, also with other conditions remaining the same of the testing group.

As a preferred embodiment of the present disclosure, the method also comprises: further cell experiments and/or animal experiments on the obtained potential substances, so as to further select and determine substances really useful for inhibiting tumors, reversing drug resistance in tumors or inhibiting/delaying senescence-associated secretory phenotype.

On the other hand, the present disclosure also provides potential substances obtained by the screening methods for inhibiting tumors, reversing drug resistance in tumors or inhibiting/delaying senescence-associated secretory phenotype. These preliminary screening substances can constitute a library for screening, so that people can finally screen for really useful drugs.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3rd ed. Science Press) or followed the manufacturer's recommendation.

### DETAILED DESCRIPTION

### Materials and Methods

### 1. Cell Culture

### (1) Cell Line Maintenance

The primary normal human prostate stromal cell line PSC27 (obtained from Fred Hutchinson Cancer Research Center, USA) was cultured in an incubator at 37° C. and 5% CO2, and proliferated and passaged in PSCC complete culture medium.

### (2) Cell Cryopreservation and Recovery

a. Cell Cryopreservation

Cells in the logarithmic growth phase were collected with 0.25% trypsin, centrifuged at 1000 rpm for 2 min. The supernatant was discarded, and the cells were resuspended in freshly prepared freezing solution. The cells were sub-packaged into labeled sterile cryovials. Then they were cooled by reducing temperature in gradient manner, and finally transferred to liquid nitrogen for long-term storage.

### B. Cell Recovery

The cells frozen in liquid nitrogen were taken out and immediately placed in a 37° C. water bath to allow them to thaw quickly. 2 mL of cell culture medium was added directly to suspend the cells evenly. After the cells adhered to the wall, fresh culture medium was used for replacement.

### (3) In Vitro Experimental Treatment

To cause cell damage, 50 μg/mL bleomycin (bleomycin, BLEO) was added to the culture medium when PSC27 cells grew to 80% (abbreviated as PSC27-CTRL). After 12 hours of drug treatment, the cells were simply washed 3 times with PBS, left in the culture medium for 7-10 days, and then the subsequent experiments were performed.

### 2. Screening of Natural Product Libraries

Pharmacodynamic analysis was conducted on an organic chemical drug library (TOPSCIENCE) with a total of 1470 small molecule compounds, mostly derived from medicinal plants and had anti-senescence potential. Each product was diluted to a 96-well plate according to a certain concentration gradient, and the density was 5000 cells per well. The medium uses DMEM, and the working concentration of natural product (or compound) is generally controlled at 1 M to 1 mM. After 3-7 days of drug treatment, cell proliferation was measured with CCK-8 Cell Counting Kit (based on WST-8 principle, Vazyme), and cell apoptosis activity was determined with Caspase 3/7 Activity Kit (Promega).

The initially identified drug candidates were further screened for 30 days. Drugs entering the second round candidate range were diluted into a 6-well plate with 20,000 cells per well. Medium and drug candidates were changed every other day. In order to determine the effect of each drug on cell phenotype and viability, etc., the confirmatory analysis was performed according to different concentrations of drugs.

### 3. Western Blotting and Immunofluorescence Detection

Cell lysate-derived proteins were separated using NuPAGE 4-12% Bis-Tris gel and transferred to nitrocellulose membranes (Life Technologies). The blot was blocked with 5% skimmed milk for 1 h at room temperature, incubated overnight at 4° C. with the desired primary antibody at the manufacturer's protocol concentration, and then incubated with horseradish peroxidase-conjugated secondary antibody (Santa Cruz) for 1 h. Blot signal detection was carried out with enhanced chemiluminescence (ECL) detection reagent (Millipore) according to the manufacturer's protocol, and ImageQuant LAS 400 Phospho-Imager (GE Healthcare) was used. As a standard protein marker, PageRuler Plus Prestained Protein Ladder (no. 26619) from Thermo Fisher Scientific was used.

For immunofluorescent staining, target cells were pre-seeded on a coverslip for at least 24 h after culture in dishes. After a brief wash, the cells were fixed with 4% paraformaldehyde in PBS for 8 min and blocked with 5% normal goat serum (NGS, Thermo Fisher) for 30 min. Mouse monoclonal antibody-anti-phospho-Histone H2A.X (Ser139) (clone JBW301, Millipore) and mouse monoclonal antibody-anti-BrdU (Cat #347580, BD Biosciences), and secondary antibody Alexa Fluor®488 (or 594)-conjugated F (ab′) 2 were sequentially added to the slides coated with the fixed cells. Nuclei were counterstained with 2 μg/ml of 4′, 6-diamidino-2-phenylindole (DAPI). The most representative image was selected from the three observation fields for data analysis and result display. FV1000 laser scanning confocal microscope (Olympus) was used to acquire confocal fluorescent images of cells.

### 4. Whole-Transcriptome Sequencing Analysis (RNA-Sequencing)

Whole-transcriptome sequencing was performed on the primary human prostate stromal cell line PSC27 under different treatment conditions. Total RNA samples were obtained from stromal cells. Their integrity was verified by Bioanalyzer 2100 (Agilent), RNA was sequenced by Illumina HiSeq X10, and gene expression levels were quantified by software package rsem (https://deweylab. github.io/rsem/). Briefly, rRNA was depleted from RNA samples with the RiboMinus Eukaryote Kit (Qiagen, Valencia, CA, USA); and according to the manufacturer's instructions, TruSeq Stranded Total RNA Preparation Kits (Illumina, San Diego, CA, USA) was used to construct a strand-specific RNA-seq library before deep sequencing.

Paired-end transcriptomic reads were mapped to a reference genome (GRCh38/hg38), and reference-annotation was performed from Gencode v27 using the Bowtie tool. Duplicate reads were identified using the picard Tools (1.98) script to mark duplicates (https://github. com/broadinstitute/picard), and only non-duplicate reads were retained. Reference splice junctions were provided by reference transcriptome (Ensembl Build 73). FPKM values were calculated with Cufflinks, and Cufflinks maximum likelihood estimation function was used to call differential gene expression. Genes with significant changes in expression were defined by false discovery rate (FDR)-corrected P-values <0.05, and only ensembl genes 73 with status “Known” and biotype “coding” were used for downstream analysis.

Next, Trim Galore (v0.3.0) (http://www.bioinformatics.babraham.ac.uk/projects/trim_galore/) was used to trim the reads, while the quality assessment was carried out by using FastQC (v0.10.0) (http://www.bioinformatics.bbsrc.ac.uk/projects/fastqc/). Subsequently, by using DAVID bioinformatics platform (https://david.ncifcrf.gov/) and Ingenuity Pathways Analysis (IPA) program (http://www.ingenuity.com/index.html), a preliminary analysis of the raw data was perform on a free online platform, Majorbio I-Sanger Cloud Platform (www.i-sanger.com) and the raw data were deposited in the NCBI Gene Expression Omnibus (GEO) database with access code GSE156448.

### 5. Protein-Protein Interaction Network Analysis

Protein-protein interaction (PPI) analysis was performed with STRING3.0. Specific proteins that met the criteria were imported into the online analysis software (http://www.networkanalyst.ca), and further hub and module analysis were performed by selecting a minimal interaction network.

### 6. Gene Set Enrichment Analysis (GSEA)

Based on the data obtained from the preliminary analysis of RNA-seq, after analyzing and comparing each differentially expressed significant gene, the genes were sorted using the “wald statistics” obtained from DESeq2. GSEA was performed on the sorting lists of all planning gene sets available in MSigDB (http://software. broadinstitute. org/gsea/msigdb). DESeq2 independent filtering was based on the average of normalized read counts to select genes with very low expression levels. The SASP and GSEA signature were as described in our previous publication (Zhang et al., 2018a).

### 7. Measurement of Gene Expression by Quantitative PCR (RT-PCR)

### (1) Extraction of Total Cellular RNA

Total RNA of cells in the growth phase or stasis phase was extracted with Trizol reagent. 1 mL Trizol was added to each T25 culture flask. The cell layer was scraped off with a cell scraper, transferred into a centrifuge tube and mixed well until not viscous. 0.2 mL of chloroform was added for every 1 ml of Trizol, shaken vigorously for 15 seconds, incubated at room temperature for 5-10 min; centrifuged at 11,000 g for 15 min at 4° C.; the colorless supernatant was transferred into a new centrifuge tube, added with 0.5 ml of isopropyl alcohol per 1 ml Trizol, incubated at room temperature for 10 min, centrifuged at 11,000 g and 4° C. for 10 minutes; the supernatant was discarded, washing was performed with 75% ethanol (at least 1 ml 75% ethanol per 1 ml Trizol was used), centrifuged at 4° C. and 7,500 g for 5 minutes; the RNA was precipitated at room temperature for 5-10 minutes (RNA could not be dried), and the precipitate was dissolved with DEPC-H2O.

After the RNA was quantified by a spectrophotometer, a small amount of total RNA was taken for 1% agarose electrophoresis to check the status and quality of the RNA.

### (2) Reverse Transcription Reaction

| | | |
|---|---|---|
| OligodT23 VN (50 uM) | 1 | ul |
| Total RNA | 1-2 | ug |

| | |
|---|---|
| RNase Free ddH2O | to 8 ul |

Above were heated at 65° C. for 5 minutes, quickly placed on ice to quench, and allowed to stand for 2 minutes.

Preparation of First-Strand cDNA Synthesis Solution

| | | |
|---|---|---|
| 2 × RT Mix | 10 | ul |
| HiScript II Enzyme Mix | 2 | ul |

The first-strand cDNA synthesis was carried out according to the following conditions:

| | | |
|---|---|---|
| 25° C. | 5 | min |
| 50° C. | 45 | min |
| 85° C. | 5 | min |

### (3) Real-Time Quantitative PCR Reaction

The reverse transcription reaction product cDNA was diluted 50 times as a template.

| | | |
|---|---|---|
| AceQ SYBR Green Master Mix | 10 | ul |
| Primer 1 (10 uM) | 0.4 | ul |
| Primer 2 (10 uM) | 0.4 | ul |
| Rox Reference Dye | 0.4 | ul |
| Template | 2 | ul |
| ddH2O to | 20 | ul |

Samples were loaded according to the above standard, and the reaction conditions were: 95° C. pre-denaturation for 15 seconds, then 95° C. for 5 seconds, 60° C. for 31 seconds, 40 cycles; melting curve conditions were 95° C. for 15 seconds, 60° C. for 30 seconds, 95° C. for 15 seconds. The sample was reacted on ABI ViiA7 (ABI) instrument. The expression of B-actin was used as an internal reference. After the reaction was completed, the amplification of each gene was checked by software analysis, the corresponding threshold cycle number was derived, and the relative expression of each gene was calculated using the 2-ΔΔCt method. The peak and waveform of the melting curve was analyzed to determine whether the obtained amplification product was a specific single target fragment.

### 8. SA-β-Gal Staining

Senescence-associated B-galactosidase (SA-β-Gal) staining was performed following a previously reported procedure (Debacq-Chainiaux, et. al., 2009). Briefly, cells in culture dishes were washed with PBS and fixed at room temperature. Cells were fixed in 2% formaldehyde and 0.2% glutaraldehyde for 3 minutes. SA-β-Gal was used for staining with freshly prepared staining solution overnight at 37° C. Images were taken on the next day and the percentage of positive cells per unit area was calculated.

### 9. Clonal Expansion Experiment

Single-cell clonal expansion experiment was performed as previously described (Duan et al., 2015; Wu et al., 2018). Briefly, cells were plated in gelatin-coated 12-well plates at a density of 2000 cells/well. Cell clones were counted after crystal violet staining.

### 10. Drug-Induced Apoptosis in Senescent Cells

PSC27 cells were plated in a 96-well dish, and the cells were induced to senescence under 50 μg/ml of BLEO treatment. SAE and ABT263 were added at concentrations of 200 μM and 1.25 μM, respectively. Cell culture medium was supplemented with Incucyte Nuclight fast Red Reagent (Essen Bioscience) and Incucyte C-3/7 Apoptosis Reagent (Essen Bioscience). Representative field of view was selected to take pictures.

### 11. Mouse Xenograft Inoculation and Preclinical Treatment Trials

All experimental mouse experiments were carried out in strict accordance with the relevant regulations of the Institutional Animal Care and Use Committee (IACUC), Shanghai Institutes for Biological Sciences, Chinese Academy of Sciences. Immuno-deficient mice (NOD-SCID mice, ICR) (body weight about 25 g) aged 6-8 weeks were used for animal experiments related to the present patent. Stromal cells PSC27 and epithelial cells PC3 were mixed at a predetermined ratio of 1:4, and each graft contained 1.25×106 cells for tissue remodeling. The xenograft tumors were implanted into mice by subcutaneous transplantation, and the animals were euthanized 8 weeks after the end of the transplantation surgery. Tumor volume was calculated according to the following formula: V=(π/6)×((1+w)/2)3 (V, volume; l, length; w, width).

In a preclinical treatment trial, subcutaneously transplanted mice were fed with a standard experimental diet, after 2 weeks chemotherapy drugs mitoxantrone (MIT, dose: 0.2 mg/kg) and/or SAE (500 μl, dose: 10 mg/kg) via intraperitoneal administration. The time points were: the former was administrated on the first day of the 3rd, 5th and 7th weeks, and the latter was administrated on the first day of the 5th and 7th weeks. A total of 3 cycles of MIT administration were carried out throughout the course of treatment, and each cycle lasted for 2 weeks. After the course of treatment, mouse tumors were harvested for volume measurement and histological analysis. Each mouse cumulatively received 0.6 mg/kg body weight of MIT and 30 mg/kg body weight of SAE. In order to cause systemic expression of SASP factors induced by chemotherapy, MIT was administered to mice through intravenous infusion according to the above steps and sequence, but the dose was reduced to 0.1 mg/kg body weight/each time (the cumulative dose of MIT received in the whole course of treatment was 0.3 mg/kg body weight) to reduce drug-related toxicity. The chemotherapy experiment ended at the end of the 8th week, and the mice were dissected immediately after sacrifice, and the xenograft tumors were collected and used for pathological system analysis.

### 12. Study on Mouse Lifespan

For the cell transplantation study, the inventors obtained 16-month-old male C57BL/6 mice by serial rearing in the SPF animal platform, with 4 to 5 animals per cage. The inventors first sorted the mice by body weight from low to high, and then selected mice with similar body weights. Next, the mice were assigned to senescence (SEN) or control (CTRL) transplant treatment at each interval using a random number generator, while the middle mice were assigned to the other treatment, so that the body weights of the senescence and control transplant mice were matched. One month after cell transplantation, when the mice were 18 months old, physical function tests were performed. After that, no further testing was done on the mice other than checking their cages. The earliest deaths occurred approximately 2 months after the last physical function test. C57BL/6 mice aged 19 to 21 months were housed at 3-5 animals per cage. As with the transplanted mice, the mice were sorted by body weight and randomly assigned to each group, and treated in either control (vehicle) or drug (SAE) group by persons blinded to the design of the preclinical trial. From the age of 24-27 months, the mice were treated with vehicle or PCC1 every 2 weeks, orally gavaged for 3 consecutive days at each time. During the course of the study, some mice were removed from their original cages in order to minimize the animal housing stress caused by long-term housing in a single cage. RotaRod and hanging tests were performed monthly because these tests were sensitive and noninvasive. At the end of the experiment, the mice were euthanized and considered to be dead if they exhibited one of the following symptoms: (i) unable to drink or eat; (ii) unwilling to move even when stimulated; (iii) rapid weight loss; (iv) severe balance disorder; or (v) body bleeding or ulcerated tumors. During the experiment, no mice were excluded due to fighting, accidental death or dermatitis. For biostatistics, a Cox proportional hazard model was used for survival analysis.

### 13. Post-Mortem Pathological Examination of Preclinical Animals

The researchers checked the cages daily and removed dead mice from the cages. Within 24 hours of animal death, cadavers (abdominal cavity, thoracic cavity and skull) were opened and kept separately in 10% formalin for at least 7 days. Decomposed or destroyed bodies were excluded. The preserved cadavers were transported to the dedicated autopsy site for pathological examination. Tumor burden (sum of different types of tumors per mouse), disease burden (sum of different histopathological changes in major organs per mouse), severity of each lesion and inflammation (lymphocyte infiltration) were assessed.

### 14. Bioluminescent Imaging

The mice were injected intraperitoneally with 3 mg of fluorescein (BioVision, Milpitas, CA), delivered in a volume of 200 μl in PBS. The mice were anesthetized with isoflurane, and bioluminescent images were acquired using a Xenogen IVIS 200 System (Caliper Life Sciences, Hopkinton, MA).

### 15. Physical Ability Test

All testing began on day 5 after the last placebo or drug treatment. Maximum walking speed was assessed using the accelerated RotaRod System (TSE System, Chesterfield, MO). The mice were trained on the RotaRod for 3 days at speeds of 4, 6 and 8 r.p.m, lasted for 200 seconds on the 1st, 2nd and 3rd days. On the test day, the mice were placed on the RotaRod, and the test was performed starting at a speed of 4 r.p.m. The rotation speed was accelerated from 4 to 40 r.p.m at intervals of 5 minutes. When the mice fell off the RotaRod, the speed was recorded. The final results were averaged from 3 or 4 trials and normalized to baseline speed. The mice that had been trained within the previous two months were no longer trained.

Forelimb grip strength (N) was measured using a Grip Strength Meter (Columbus Instruments, Columbus, OH), and the results were averaged from more than 10 trials. For the hanging endurance test, the mice were placed on a 2 mm thick metal wire, while the later was 35 cm above a mat. The mice were only allowed to grasp the wire with their forelimbs, and the hang time was normalized to body weight and expressed as hang duration (sec)×body weight (g). The results were the average of 2 to 3 trials per mouse. Their daily activity and food intake were monitored for 24 h (12 hours of light and 12 hours of dark) by a Comprehensive Laboratory Animal Monitoring System (CLAMS). The CLAMS system was equipped with an Oxymax Open Circuit calorimeter System (Columbus Instruments). For treadmill performance, the mice were acclimatized to run on an electric running machine (Columbus Instruments) at a 5° incline, for 3 days of training, lasted for 5 minutes each day, started at a speed of 5 m/min for 2 minutes, then accelerated to 7 m/min for 2 minutes, then 9 m/min for 1 minute. On the day of the test, the mice ran on the treadmill at an initial speed of 5 m/min for 2 min, and then the speed increased by 2 m/min every 2 min until the mice were exhausted. Fatigue was defined as the inability of mice to return to the treadmill despite mild electrical and mechanical stimulation. The distance was recorded after the test, and the total work (KJ) was calculated with the following formula:

$$mass ( kg ) \times g ( 9.8 m / s 2 ) \times distance ( m ) \times sin ( 5 ° ) .$$

### 16. Biostatistical Methods

In the present patent application, all in vitro experiments involving cell proliferation rate, survival rate and SA-β-Gal staining, and in vivo experiments on mouse xenograft tumors and preclinical drug treatment were repeated more than 3 times, and the data were presented in the form of mean±standard error. The statistical analyzes were established on the basis of raw data and calculated by one-way analysis of variance (ANOVA) or a two-tailed Student's t-test, while the results with P<0.05 were considered to be significantly different.

The correlation between factors was tested by Pearson's correlation coefficients. Cox proportional hazards model was used for survival analysis when mice were obtained in several cohorts and grouped in cages. In the model, sex and age were used in the treatment as fixed effects, while cohort and initial cage assignment were used as random effects. Since during the study some mice were moved from their initial cages to minimize stress from the single cage enclosure, we also per-formed analysis without cage effects. The results of the two analyzes did not differ significantly in directionality or statistical significance, which increased confidence in results. Survival analysis was performed using the statistical software R (version 3.4.1; library “coxme”). In most experiments and outcome assessments, investigators made blind selections for assignments. The inventors used baseline body weights to assign mice to experimental groups (to achieve similar body weights between groups), so randomization was only performed within groups matched for body weight. The sample size was determined based on previous experiments and therefore did not use statistical power analysis. All replicates in this study were derived from different samples, and each sample was derived from a different experimental animal.

### Example 1: SAE can Effectively Inhibit SASP Expression when Used at a Low Concentration

1.SAE can Effectively Inhibit SASP Expression when Used at a Low Concentration.

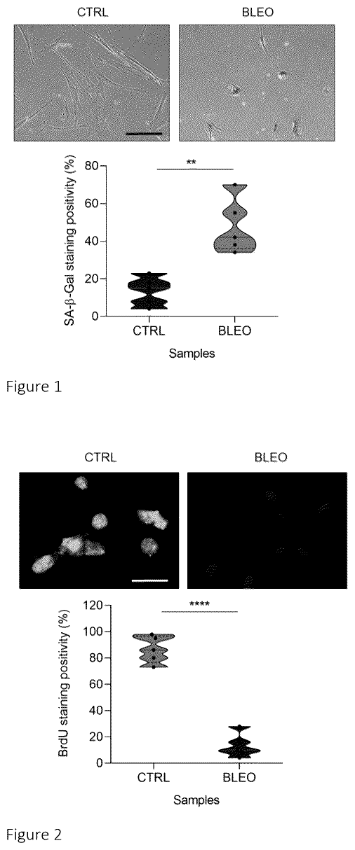

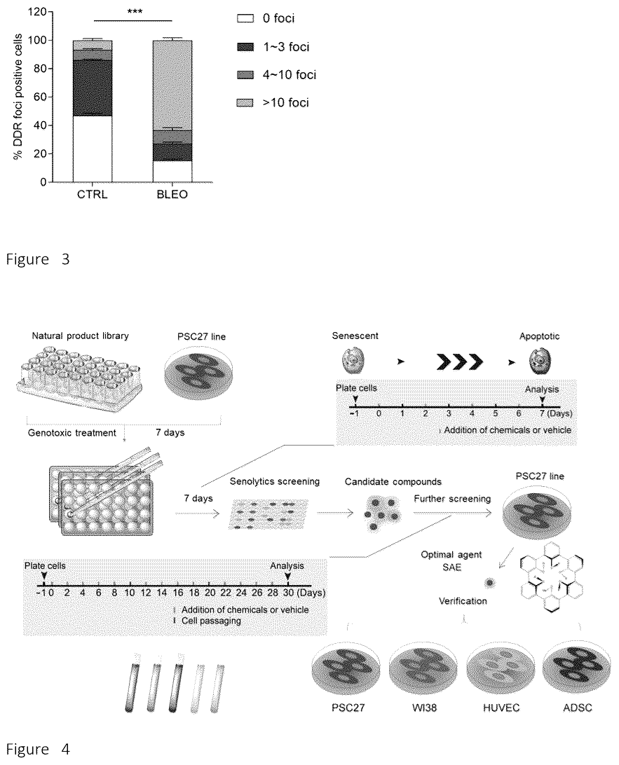

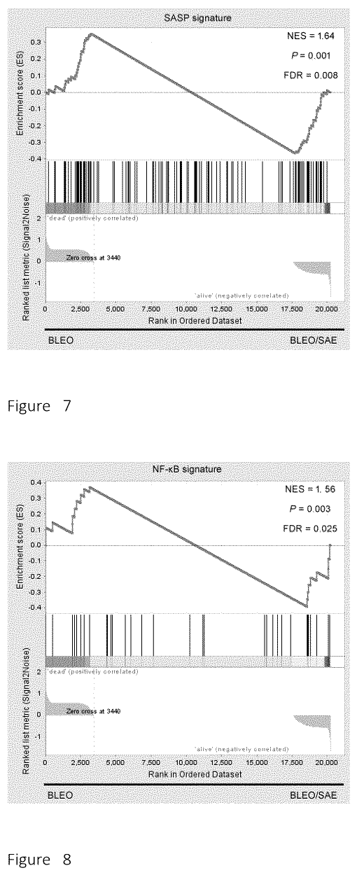

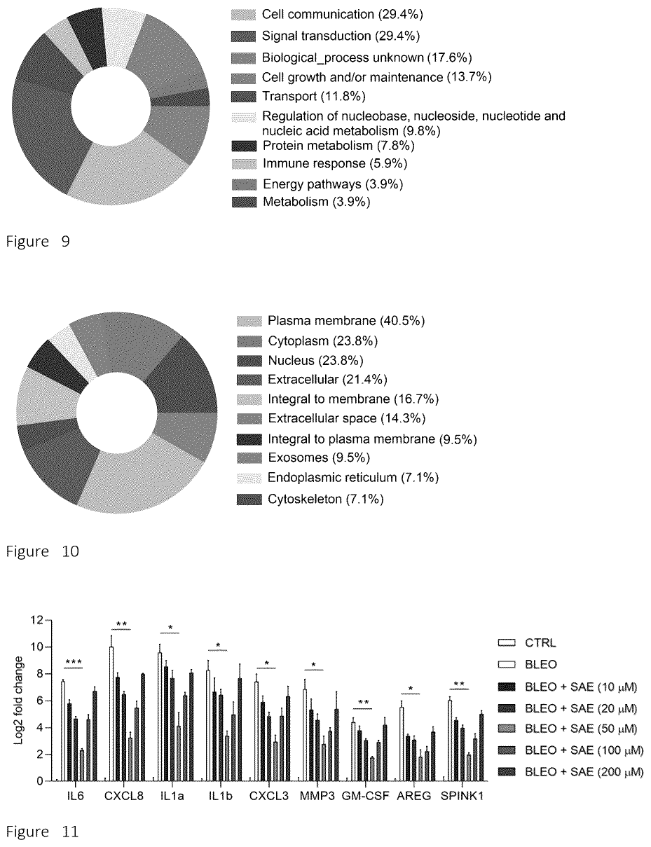

To identify innovative compounds capable of effectively modulating phenotype of senescent cells, an unbiased screen was performed using an organic chemical drug library composed of 1470 small molecules. In order to test the efficacy and potential biological value of these drugs, the primary normal human prostate stromal cell line, PSC27, was chosen to be used as an in vitro cell model. PSC27 is composed predominantly of fibroblasts, also with non-fibroblastic cell lines (including endothelial cells and smooth muscle cells), but in smaller proportions; and PSC27 is a human primary stromal cell line in nature, which forms typical SASP after exposure to stress factors such as genotoxic chemotherapy or ionizing radiation. By using the methods optimized in the preliminary experiment, these cells were treated with a specific dose of bleomycin (BLEO). A significant increase in the positive rate of senescence-associated β-galactosidase (SA-β-Gal) staining, a greatly reduced BrdU incorporation rate, and a significantly increased DNA damage repair foci (DDR foci) within days after drug injury were observed (FIG. 1 to FIG. 3). A systematic screening was performed to compare the effects of these natural drug products on the expression profiles of senescent cells in parallel mode (FIG. 4). RNA-seq was carried out on these cells. Subsequently, the high-throughput data obtained showed that a small molecular compound, salvianolic acid E (SAE), significantly altered the expression profile of senescent cells (FIG. 5). Among them, thousands of genes were significantly down-regulated, while many genes were up-regulated at the same time. Importantly, the expression of SASP factors was generally decreased in senescent cells after SAE treatment, whereas these SASP factors were generally significantly up-regulated in senescent cells (FIG. 6). Although the expression profiles of some SASP-unrelated genes showed similar trends to those of typical SASP factors, the data from the GSEA analysis further revealed significant repression of molecular signatures characterizing SASP expression or NF-κB activation, which were major transcriptional event mediating the development of proinflammatory SASP (FIG. 7, FIG. 8). Further GO bioinformatics data revealed that these molecules are functionally involved in a set of important biological processes, including signal transduction, intercellular communication, cellular metabolism, energy regulation, nucleotide metabolism and protein metabolism (FIG. 9). Most of these down-regulated genes, in addition to playing a certain role in the nucleus, were proteins in nature that were released into the extracellular space after expression, or were located on the plasmic membrane or in the cytoplasm, and generally corresponded characteristically to the secretory properties of these molecules (FIG. 10).

To further confirm the effect of SAE on SASP expression under in vitro conditions, the PSC27 cells were treated under a series of in vitro concentration gradients. The data showed that SAE at a working concentration of 50 μM inhibited SASP development with the greatest efficiency (FIG. 11). However, lower or higher concentrations of the drug were less effective, although the latter could be related to the cellular stress response caused by the drug's increased cytotoxicity (FIG. 11). Therefore, SAE, a plant-derived natural small molecular compound, could be used to control the pro-inflammatory phenotype of senescent cells, namely SASP, especially at relatively low concentrations.

### Example 2: SAE as a Novel Senolytic Agent when Used at High Concentrations

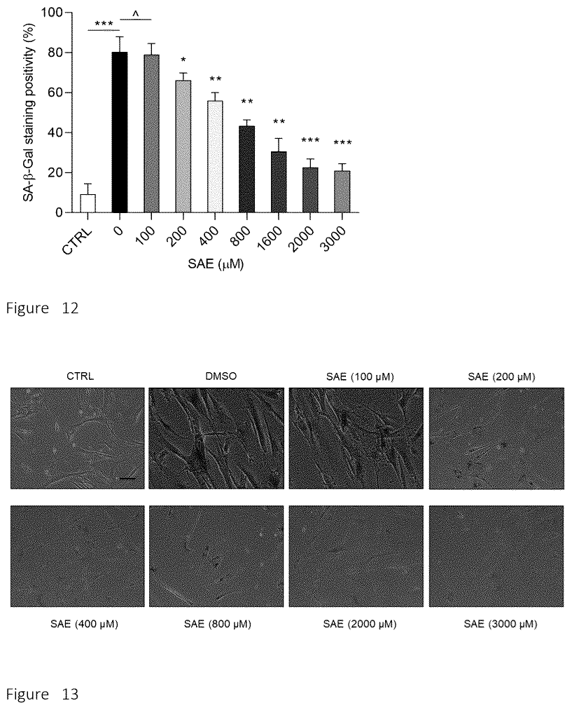

Given the remarkable efficacy of SAE in controlling SASP expression, the potential of this natural product was then explored in killing senescent cells at higher concentrations. Therefore, the percentage survival of treated senescent cells under in vitro conditions were measured with increasing concentrations of SAE. SA-β-Gal staining data indicated that senescent cells were not eliminated until the SAE concentration reached 200 μM (FIG. 12). As the concentration increased, the killing effect of SAE on senescent cells (80% staining positive) was further enhanced, and a threshold was reached when SAE was 2000 μM (20% of senescent cells remained at this time); when its concentration was increased to 3000 μM, the killing effect of SAE was not further enhanced (FIG. 12; FIG. 13).

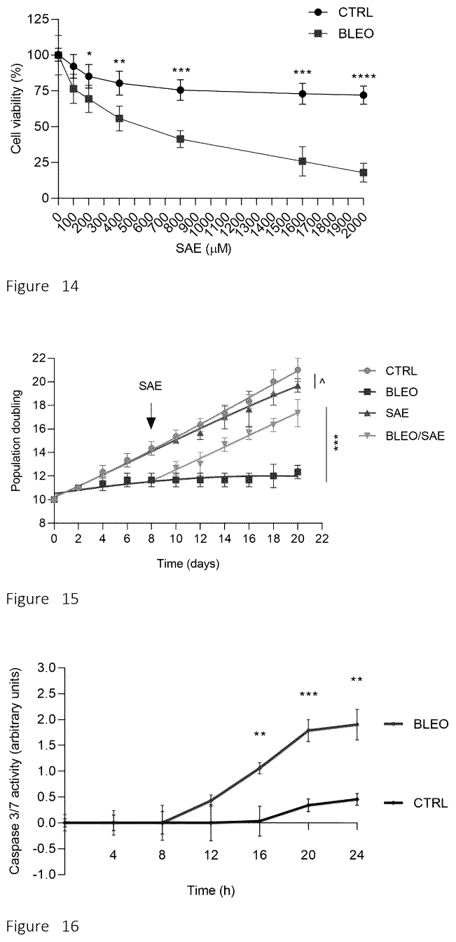

To further dissect these issues, the inventors conducted confirmatory experiments. Cell viability assays showed that SAE induced significant death of senescent cells starting from a concentration of 200 μM as compared to the corresponding control proliferating cells (FIG. 14). When the SAE concentration was increased to 2000 μM, the percentage of surviving senescent cells decreased to approximately 10%. However, even at 3000 μM of SAE, proliferating cells were not significantly reduced. These results confirmed the high selectivity and outstanding specificity of SAE to senescent cells, and this feature was actually the basic technical requirement for senolytics as a unique class of anti-senescent drugs in the world.

The inventors next investigated the population doubling (PD) potential of stromal cells following genotoxic treatment. Compared with the cells of the BLEO group that entered a state of growth arrest rapidly after injury treatment, the group treated with a combination of BLEO and SAE showed a significantly increased PD ability (FIG. 15). Interestingly, however, SAE itself does not affect PD in proliferating cells, and this data further demonstrates that SAE is selective and target-specific between senescent cells and normal cells.

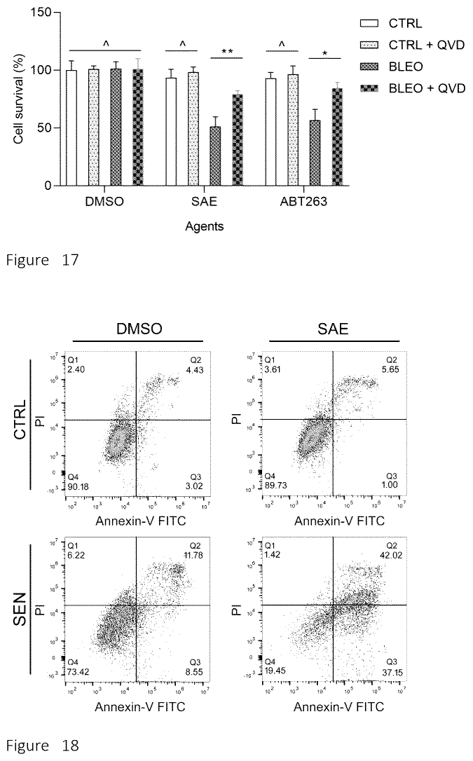

In order to explore whether SAE caused senescent cells to lose their viability by inducing apoptosis, the inventors used SAE to treat the proliferation group cells and the senescent group cells respectively under culture conditions. The subsequently observed changes in caspase-3/7 activity showed that SAE caused apoptosis of senescent cells; from the 16th hour after the addition of SAE, there was a statistical difference between the senescent group and the control group (FIG. 16). In addition, the pan-caspase inhibitor QVD could prevent the killing of senescent cells by SAE, and the actual effect in this process was very similar to the effect of ABT263 (a currently known and very effective inducer of apoptosis in senescent cells) on senescent cells (FIG. 17). The above series of results confirmed that SAE promoted senescent cells to enter the death program by inducing apoptosis, but proliferating cells were basically not targeted or affected by this natural drug.

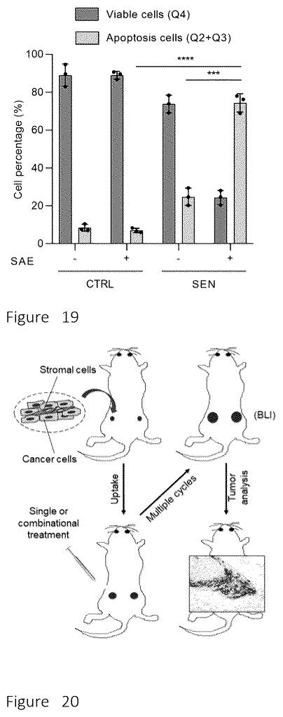

Given the apparent effect of SAE on senescent cells, the inventors then analyzed the potential of SAE to induce apoptosis. Flow cytometry data showed that the viability of senescent PSC27 cells was significantly reduced, while the proportion of apoptosis was significantly increased, but the change of proliferating cells was not obvious (FIG. 18; FIG. 19). Thus, the consistency of inventors 1 data supported that SAE caused the elimination of senescent cells by inducing apoptosis in vitro and this natural product had an outstanding potential in targeting senescent cells.

### Example 3: Use of SAE to Therapeutically Target Senescent Cells to Promote Tumor Regression and Effectively Reduce Chemotherapy Resistance

In view of the outstanding selectivity of SAE in clearing senescent cells at high concentrations in vitro, then it was next considered whether this drug could be used to intervene in various age-related diseases in vivo. Cancer is one of the major chronic diseases that seriously threaten human life and endanger health. In addition, drug resistance of cancer cells limits the effect of most anticancer treatments in clinic, and senescent cells often promote the occurrence of therapeutic drug resistance of surrounding cancer cells by developing SASP in damaged tumor foci. Even so, the feasibility and safety of removing senescent cells from primary tumors to promote a therapeutic index of cancer has been largely unexplored to date.

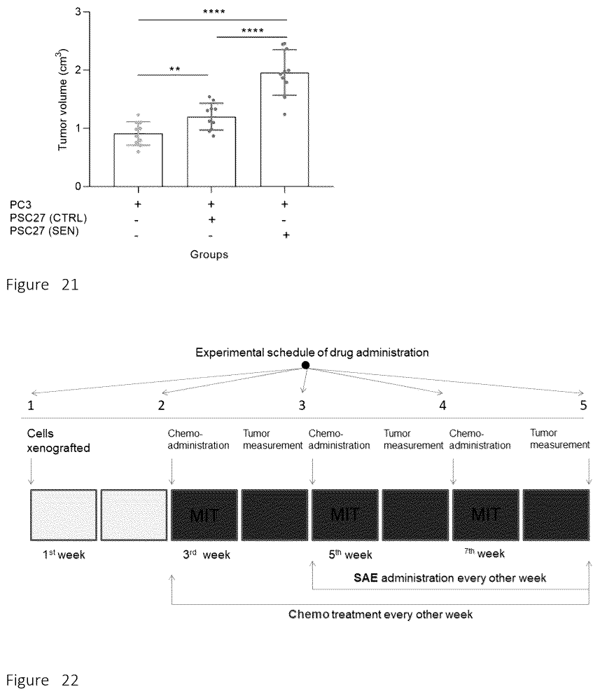

First, the inventors constructed tissue recombinant by mixing PSC27 stromal cells with PC3 epithelial cells, the latter is a typical highly malignant prostate cancer cell line. Before the recombinant was implanted subcutaneously in the posterior thigh of non-obese diabetic and severe combined immunodeficiency (NOD/SCID) mice, the ratio of stromal cells to epithelial cells was 1:4. Tumor size (volume) was measured at the end of 8 weeks after recombinant implantation in animals (FIG. 20). Compared with tumors composed of PC3 cancer cells and primary PSC27 stromal cells, the volumes of xenografts composed of PC3 cells and senescent PSC27 cells were significantly increased (P<0.0001), this difference further confirmed the critical facilitative role of senescent cells in tumor progression (FIG. 21).

To more closely approximate clinical conditions, the inventors specifically designed a preclinical protocol involving genotoxic chemotherapeutic drug treatment and/or senescent drug intervention (FIG. 22). Two weeks after subcutaneous implantation, when stable tumor uptake in vivo was observed, experimental animals were provided with a single dose of MIT (Mitoxantrone, a chemotherapeutic agent) or placebo at the first day of the 3rd, 5th, 7th weeks until the end of the 8 weeks regimen. MIT administration significantly delayed tumor growth compared to the placebo-treated group, that confirmed the efficacy of MIT as a chemotherapeutic agent (44.1% reduction in tumor size, P<0.0001) (FIG. 23). Notably, although SAE itself did not cause tumor shrinkage, the administration of SAE significantly reduced tumor size in mice treated with MIT (32.0% reduction in tumor volume compared with MIT, P<0.01; 62.0% reduction in tumor volume compared with placebo treatment, P<0.0001) (FIG. 23).

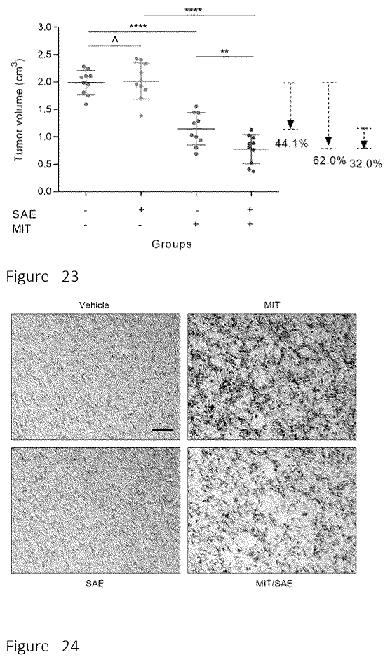

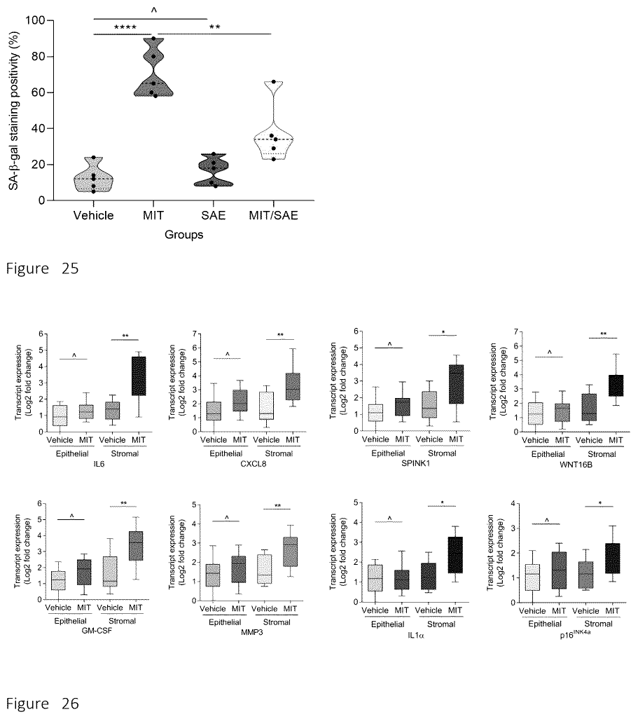

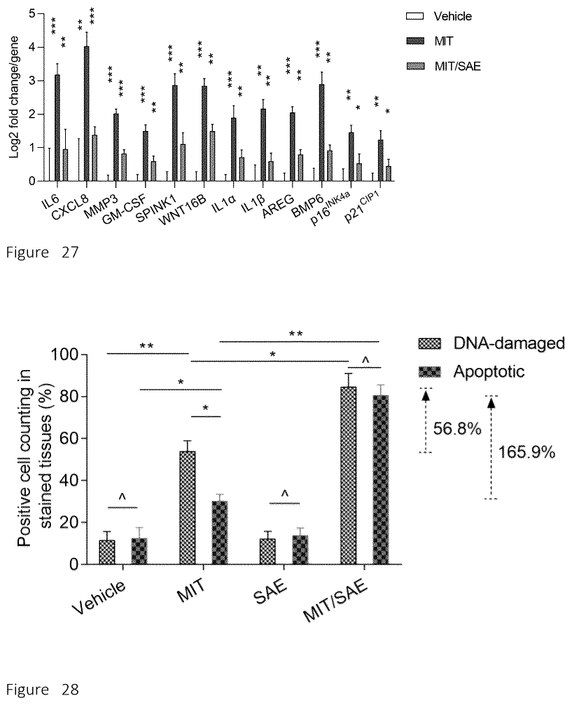

Next, the inventors inferred whether cell senescence occurred in tumor lesion in these animals. The test results proved that the MIT administration process induced the appearance of a large number of senescent cells in the tumor tissue, although this was not surprising. However, SAE administration essentially depleted most senescent cells in the lesions of these animals treated with chemotherapeutic drugs (FIG. 24; FIG. 25). Results of laser capture microdissection (LCM) and subsequent quantitative PCR revealed significantly elevated expression of SASP factors, including IL6, CXCL8, SPINK1, WNT16B, GM-CSF, MMP3, IL1A, and this trend accompanied with the up-regulation of senescence marker p16INK4A in chemotherapeutic animals (FIG. 26). Interestingly, these changes occurred mainly in stromal cells, but not in their adjacent cancer cells, implying the possibility of repopulation of residual cancer cells, the residual cancer cells had developed acquired resistance in the treatment-damaged tumor microenvironment (TME). However, this change was largely reversed when SAE was administered, as demonstrated by analysis of transcript-level data (FIG. 27).

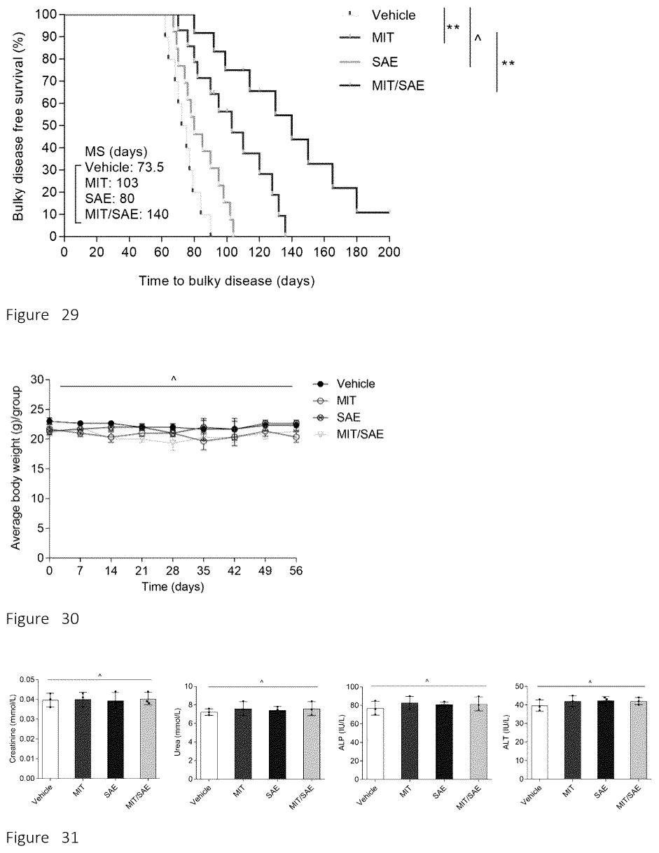

To investigate mechanisms directly supporting this senescence-associated pattern with expression and reversal of SASP in MIT-administered mice, the inventors dissected tumors in animals treated with both drugs 7 days after the first SAE administration, and the time point of 7 days after administration was selected mainly because drug-resistant clones had not yet formed in cancer cells in the lesion at this time. Compared with placebo, MIT administration resulted in a significant increase in both the degree of DNA damage and apoptosis. Although SAE alone could not induce DNA damage or cause apoptosis, the chemotherapeutic drug MIT could highly up-regulate these two indicators (FIG. 28). However, when MIT-treated animals were administered with SAE, the indices of DNA damage or apoptosis were significantly enhanced, implying the enhanced cytotoxicity at tumor sites in these senescent drug-treated animals. As supporting evidence, when SAE was applied during treatment, caspase 3 cleavage activity was increased, which was a typical hallmark of apoptosis (FIG. 29).

Then the survival of animals in different drug-treated groups was compared, primarily in a time-prolonged manner to assess the consequences of tumor progression. In this preclinical cohort, animals were monitored for prostate tumor growth, and severe disease was judged to have occurred once the endosomal tumor burden in mice was prominent (size≥2000 mm3), which was a method used for assessing progression of tumors and other diseases in certain conditions. The mice treated with the MIT/SAE combination exhibited the longest median survival, prolonging the survival by at least 35.9% compared to the group treated with MIT alone (FIG. 29, green versus blue). However, treating tumor-bearing mice with SAE alone did not result in a significant benefit, with only a marginal extension of survival.

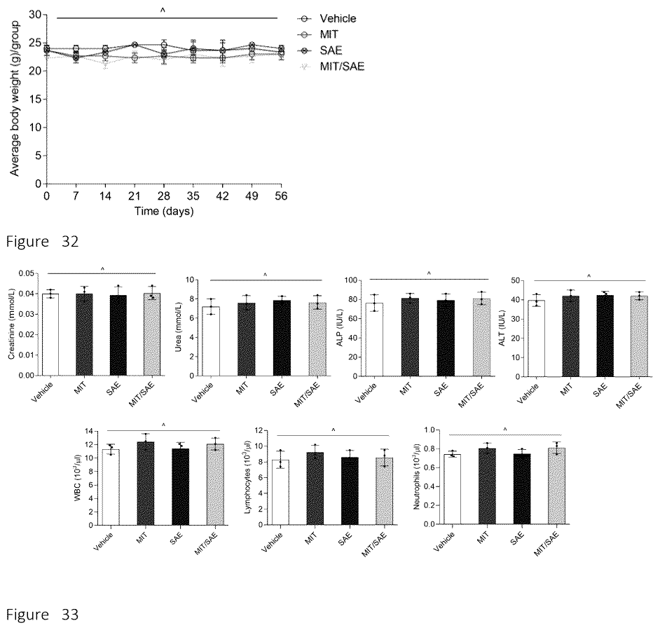

Remarkably, the treatments administered in these studies appeared to be well tolerated by the experimental mice. Significant fluctuations in urea, creatinine, alkaline phosphatase, glutamic-pyruvic transaminase, or body weight were not observed (FIG. 30; FIG. 31). More importantly, chemotherapy and anti-senescent drugs used at each drug dose designed for this study did not significantly interfere with the integrity of the immune system and tissue homeostasis of key organs, even in immune competent wild-type mice (FIG. 32; FIG. 33). These results consistently demonstrate that the combination of anti-senescent agents with conventional chemotherapeutic agents has the potential to enhance tumor response in a general sense without causing severe systemic toxicity.

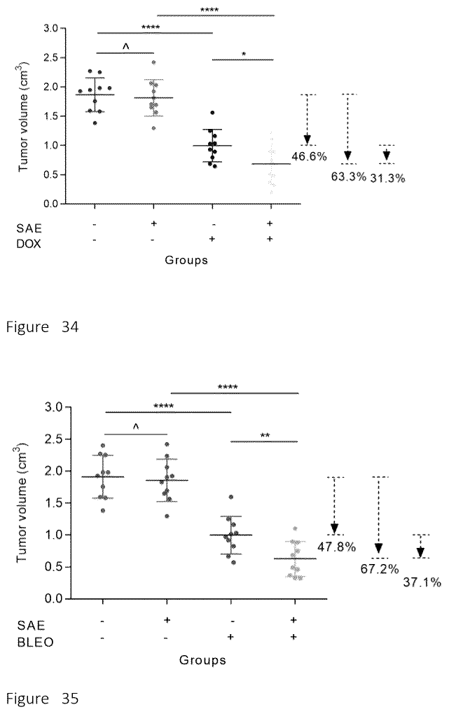

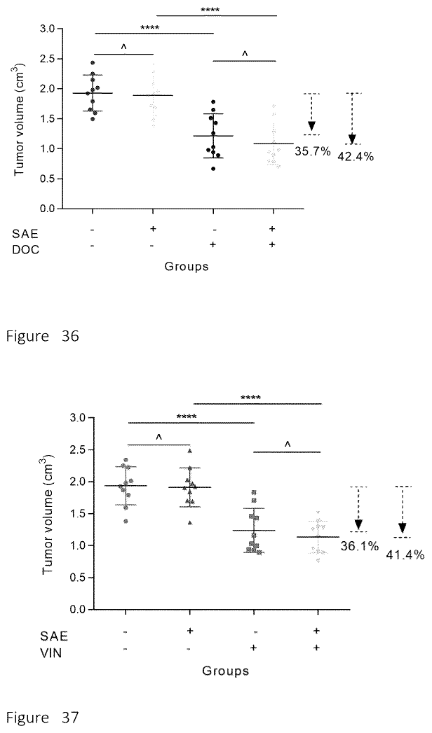

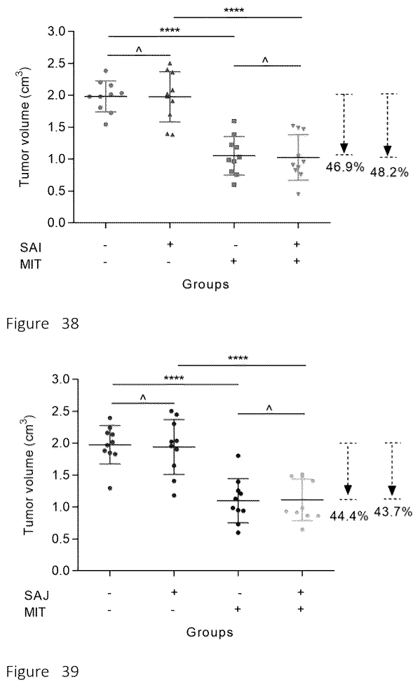

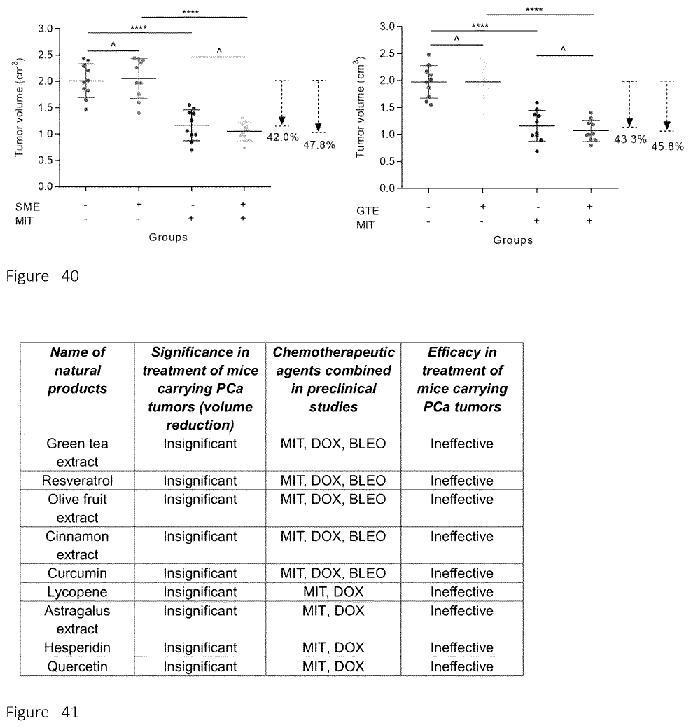

In order to determine whether SAE was drug-dependent or specific in improving the effect of chemotherapy, doxorubicin (DOX), docetaxel (DOC) and vincristine (VIN) were then chosen to be used to combine with SAE respectively for preclinical trials. The results showed that among these chemotherapeutic drugs, only the two combinations of DOX and SAE, BLEO and SAE could roughly repeat the significant effect caused by the combined treatment of MIT and SAE (FIG. 34; FIG. 35). However, although DOC and VIN could reduce the tumor volume when used alone, no further tumor shrinkage achieved when any one of DOC and VIN was administered together with SAE, that was, no more benefits can be realized (FIG. 36, FIG. 37). Therefore, the characteristic of SAE enhancing the therapeutic effect of chemotherapy under in vivo conditions is limited to its combination with specific genotoxic drugs and it is dependent on the type of drugs. Notably, other molecules from the salvianolic acid family, such as salvianolic acid I (SAI) and salvianolic acid J (SAJ), showed no significant effects when combined with MIT (FIG. 38, FIG. 39). Additionally, combining the extract of Danshen (Salvia miltiorrhiza extract, SME) containing various molecules from the salvianolic acid family with MIT did not produce significant effects (FIG. 40). Similarly, using green tea extract (GTE) with typically certain anti-senescent effects in the human body, in combination with MIT also failed to yield significant therapeutic results (FIG. 40). These results indicate that SAE possesses unique biological activity and targeting senescent cells within the entire salvianolic acid family, making it exceptionally valuable for future interventions in senescence in the body and the control of senescence-associated diseases. When various extracts from different natural plant sources or small-molecule compounds derived from secondary plant metabolites are used in similar animal experiments, they generally fail to achieve effective therapeutic results in combination with chemotherapy drugs for tumor treatment (FIG. 41).

### Example 4: Clearance of Senescent Cells by SAE Treatment Prolonging Late-Life Survival in Aged Mice without Increasing Morbidity in Late-Life Stage

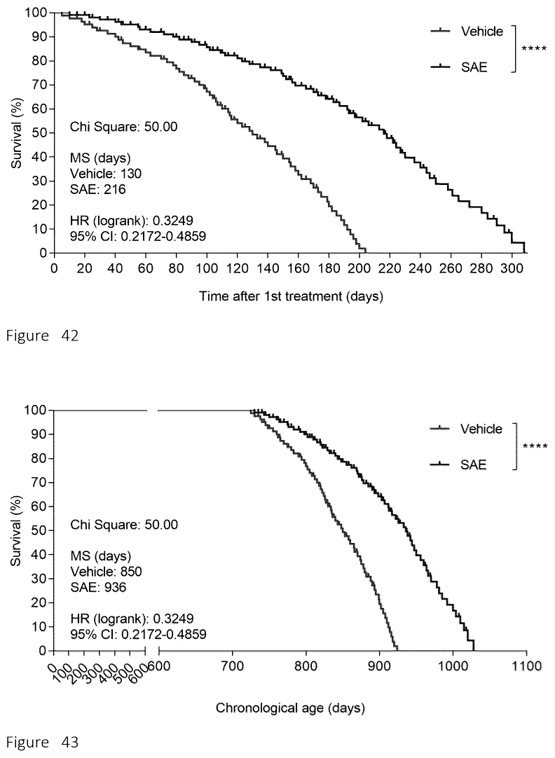

Since SAE has the amazing efficacy of clearing senescent cells, reducing tumor drug resistance and improving the overall therapeutic effect in the microenvironment of tumor mice, is there also some significant benefit in promoting health or delaying disease in naturally aging animals? To answer this question, firstly, it should be considered whether a potentially translational approach could be used to eliminate senescent cells, that was, intermittent treatment from a very old point in time whether extend the remaining lifespan of WT mice? In this regard, a series of in vivo experiments were carried out accordingly. It was worth noting, and quite surprising, that in the SAE group administered from the age of 24-27 months (equivalent to the age of 75-90 years in humans) under the regimen of taking the drug every two weeks, the median survival period after treatment was 66.2% longer than that of the vehicle group, and it had a lower risk of death (HR=0.3249, SA group/vehicle group; P<0.0001) (FIG. 42, FIG. 43). This finding suggests that SAE-mediated clearance of senescent cells can reduce the risk of death in aged mice and effectively prolong their survival.

Above-described examples only show several embodiments of the present disclosure, which are described specifically and in detail. However, it should not be understood as a limiting patent scope of the present disclosure. It should be noted that those skilled in the art can make some adjustments and improvements without departing from the concept of the present disclosure and all these forms are within the scope of protection of the present disclosure. Therefore, the scope of patent protection in the present disclosure should be determined by the appended claims. Simultaneously, each reference provided herein is incorporated by reference to the same extent as if each reference was individually incorporated by reference.

1. A method for specifically-targeted clearance of senescent cells in a tumor microenvironment and tumor inhibition, comprising administering to a subject in need of treatment with a combination of a salvianolic acid E compound or a pharmaceutically acceptable salt, ester, isomer, solvate or prodrug thereof, and a chemotherapeutic drug; wherein the chemotherapeutic drug is capable of inducing a senescence-associated secretory phenotype in the tumor microenvironment after administration.

2. The method according to claim 1, wherein, the tumor is a tumor that exhibits a senescence-associated secretory phenotype in the tumor microenvironment after treatment with genotoxic drugs and/or a tumor that develops drug resistance after treatment with genotoxic drugs.

3. The method according to claim 2, wherein, the tumor comprises: prostate cancer, breast cancer, lung cancer, colorectal cancer, gastric cancer, liver cancer, pancreatic cancer, bladder cancer, skin cancer, kidney cancer, esophageal cancer, bile duct cancer, brain cancer.

4. The method according to claim 1, wherein, the senescence-associated secretory phenotype is a senescence-associated secretory phenotype caused by DNA damage; preferably, the DNA damage is a DNA damage induced by a chemotherapeutic drug.

5. The method according to claim 1, wherein, the chemotherapeutic drug is a genotoxic drug; more preferably, comprising: mitoxantrone, doxorubicin and bleomycin.

6. The method according to claim 5, wherein, the chemotherapeutic drug is mitoxantrone, wherein the weight ratio of mitoxantrone to salvianolic acid E is 1:20˜80; or
the chemotherapeutic drug is bleomycin, with a final concentration of 30˜70 μg/mL, and the salvianolic acid E has a final concentration of 200˜550 μM; or
the chemotherapeutic drug is bleomycin, with a final concentration of 30˜70 μg/ml, and the salvianolic acid E has a final concentration of 700˜5000 μM; or
the chemotherapeutic drug is doxorubicin, wherein the weight ratio of doxorubicin to salvianolic acid E is 1:4 to 1:16.

7. A method for inhibiting senescence, prolonging lifespan or prolonging the survival period of the old or inhibiting a senescence-associated secretory phenotype, comprising administering to a subject in need of treatment with salvianolic acid E or a pharmaceutically acceptable salt, ester, isomer, solvate or prodrug thereof.

8. A composition, comprising: (a) salvianolic acid E or a pharmaceutically acceptable salt, ester, isomer, solvate or prodrug thereof, and (b) a chemotherapeutic drug, with a capacity of inducing a senescence-associated secretory phenotype in the tumor microenvironment after administration.

9. The composition according to claim 8, wherein, the chemotherapeutic drug is a genotoxic drug; more preferably, comprising: mitoxantrone, doxorubicin and bleomycin.

10. The composition according to claim 8, wherein the composition also comprises a pharmaceutically acceptable auxiliary material.

11. The composition according to claim 8, wherein, the chemotherapeutic drug is mitoxantrone, wherein the weight ratio of mitoxantrone to salvianolic acid E is 1:20˜80; or the chemotherapeutic drug is bleomycin, with a final concentration of 30˜70 μg/ml, and the salvianolic acid E has a final concentration of 200˜550 μM; or
the chemotherapeutic drug is bleomycin, with a final concentration of 30˜70 μg/mL, and the salvianolic acid E has a final concentration of 700˜5000 μM; or
the chemotherapeutic drug is doxorubicin, wherein the weight ratio of doxorubicin to salvianolic acid E is 1:4 to 1:16.

12. A drug kit for specifically-targeted clearance of senescent cells in a tumor microenvironment and tumor inhibition, comprising the composition according to claim 8.

13. A method for preparing a composition or a drug kit for inhibiting tumor, comprising mixing the salvianolic acid E compound or a pharmaceutically acceptable salt, ester, isomer, solvate or prodrug thereof and the chemotherapeutic drug; or placing the salvianolic acid E compound or a pharmaceutically acceptable salt, ester, isomer, solvate or prodrug thereof and the chemotherapeutic drug in the same drug kit.

14. The method according to claim 13, wherein, the chemotherapeutic drug is a genotoxic drug; more preferably, comprising: mitoxantrone, doxorubicin and bleomycin.

15. A method for screening a potential substance for promoting salvianolic acid E to clear senescent cells in a tumor microenvironment, inhibit tumor, or prolong the lifespan, wherein the method comprises:
(1) providing a system of tumor microenvironment, wherein the system comprises tumor cells and stromal cells;
(2) treating the system of (1) with a chemotherapeutic drug, inducing a senescence-associated secretory phenotype in the tumor microenvironment and administering salvianolic acid E before, during or after inducing the senescence-associated secretory phenotype in the tumor microenvironment;
(3) adding the candidate substance to the system in (2) and observing its effect on the tumor microenvironment system; if the candidate substance statistically promotes salvianolic acid E to clear senescent cells in a tumor microenvironment, then the candidate substance is a potential substance that can be used in combination with salvianolic acid E to clear senescent cells in the tumor microenvironment or inhibit tumors or prolong the lifespan.

16. The method according to claim 15, wherein, cell apoptosis or the status of the senescence-associated secretory phenotype (SASP) can be evaluated by observing caspase-3/7 activity or the expression of SASP factors; or cell apoptosis or the status of the senescence-associated secretory phenotype (SASP) can be evaluated by observing senescence marker p16INK4A in animals administering with a chemotherapeutic drug.

17. The method according to claim 16, wherein, the SASP factors comprise: IL6, CXCL8, SPINK1, WNT16B, GM-CSF, MMP3, CXCL1, CXCL3, IL-1a, IL-1B.

18. A method for screening a potential substance for inhibiting senescence-associated secretory phenotype, wherein the method comprises:
(1) providing a system of stromal cells, inducing the senescence-associated secretory phenotype in the system; administering salvianolic acid E before, during or after inducing the senescence-associated secretory phenotype in the system;
(2) adding the candidate substance to the system of (1) and observing its effect on the system of stromal cells; if the candidate substance statistically promotes salvianolic acid E to inhibit the senescence-associated secretory phenotype, then the candidate substance is a potential substance that can be used in combination with salvianolic acid E to inhibit the senescence-associated secretory phenotype.
